(19) United States
(12) Patent Application Publication
(10) Pub. No.: US 2023/0241373 A1
(43) Pub. Date: Aug. 3, 2023

(54) WEARABLE CARDIAC THERAPEUTIC DEVICES WITH HYDROPHOBIS AND/OR HYDROPHILIC DIELECTRIC FIBERS

(71) Applicant: ZOLL Medical Corporation, Chelmsford, MA (US)

(72) Inventors: Christopher Lawrence Swenglish, Connellsville, PA (US); Saundra L. Penkunas, Portland, OR (US)

(21) Appl. No.: 18/100,100

(22) Filed: Jan. 23, 2023

Related U.S. Application Data
(60) Provisional application No. 63/305,539, filed on Feb. 1, 2022.

(51) Int. Cl. A61N 1/04; A61N 1/39

(52) U.S. Cl. CPC A61N 1/0404; A61N 1/3904; A61B 5/25

(57) ABSTRACT

A wearable cardiac therapeutic device includes at least one therapy electrode configured to deliver therapeutic electrical pulses to a patient's heart; and a support garment including at least one support pocket for supporting the at least one therapy electrode including a mesh interface including: a first side including hydrophobic fiber(s) proximate to electrically conductive fluid deployment opening(s) on the therapy electrode; a second side including hydrophilic fiber(s) proximate to the patient's skin; and conductive fiber(s) and/or conductive particles configured to be interspersed with the hydrophobic fiber(s) and hydrophilic fiber(s) such that the conductive fiber(s) and/or conductive particles conduct therapeutic electrical current from the therapy electrode to the patient's skin, wherein the mesh interface is configured to transfer electrically conductive fluid dispersed from one or more electrically conductive fluid reservoirs disposed on the therapy electrode towards the patient's skin.

### CROSS REFERENCE TO RELATED APPLICATIONS

This application claims priority to U.S. Provisional Application No. 63/305,539 filed Feb. 1, 2022 entitled “Therapy Electrode Mesh Interface for Wearable Cardiac Therapeutic Devices,” the entire contents of which is incorporated by reference herein.

### FIELD OF THE DISCLOSURE

The present disclosure relates to a support garment for a wearable cardiac monitoring and therapeutic medical device, such as a wearable cardioverter defibrillator (WCD).

### BACKGROUND OF THE DISCLOSURE

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

When a patient is deemed at high risk of death from arrhythmias, such as ventricular fibrillation or ventricular tachycardia, electrical devices can be implanted so as to be readily available when treatment is needed. However, patients who have recently had a heart attack or are awaiting such an implantable device can be kept in a hospital where corrective electrical therapy is generally close at hand. Long-term hospitalization is frequently impractical due to its high cost, or due to the need for patients to engage in normal daily activities.

Wearable cardioverter defibrillators can help bridge the gap for patients who have recently experienced cardiac arrest, who are susceptible to heart arrhythmias and are at temporary risk of sudden death, and/or who are awaiting an implantable device. Support garments have been developed for supporting the components of such wearable cardioverter defibrillators, including the sensing and therapeutic energy delivery electrodes, such that the electrodes are properly positioned against the patient's skin. Such support garments can incorporate a material that acts as an interface between the therapeutic energy delivery electrodes and the patient's skin.

### SUMMARY OF SOME OF THE EMBODIMENTS

Non-limiting examples of embodiments will now be described.

In some examples, a mesh interface for use with a support garment of a wearable cardiac therapeutic device is provided, the mesh interface comprising: a first side comprising hydrophobic dielectric fiber(s) proximate to one or more electrically conductive fluid deployment openings on a therapy electrode; a second side comprising hydrophilic dielectric fiber(s) proximate to the patient's skin; and conductive fiber(s) and/or conductive particles configured to be interspersed with the hydrophobic dielectric fiber(s) and with the hydrophilic dielectric fiber(s), such that the conductive fiber(s) and/or conductive particles conduct therapeutic electrical current from the therapy electrode to the patient's skin, wherein the mesh interface is configured to facilitate transfer of electrically conductive fluid from one or more electrically conductive fluid reservoirs disposed on the therapy electrode through the one or more electrically conductive fluid deployment openings of the therapy electrode and towards the patient's skin.

In some examples, a support garment of a wearable cardiac therapeutic device is provided, the support garment comprising a mesh interface comprising: a first side comprising hydrophobic dielectric fiber(s) proximate to one or more electrically conductive fluid deployment openings on a therapy electrode; a second side comprising hydrophilic dielectric fiber(s) proximate to the patient's skin; and conductive fiber(s) and/or conductive particles configured to be interspersed with the hydrophobic dielectric fiber(s), and with the hydrophilic dielectric fiber(s) such that the conductive fiber(s) and/or conductive particles conduct therapeutic electrical current from the therapy electrode to the patient's skin, wherein the mesh interface is configured to facilitate transfer of electrically conductive fluid from one or more electrically conductive fluid reservoirs disposed on the therapy electrode through the one or more electrically conductive fluid deployment openings of the therapy electrode and towards the patient's skin.

In some examples, a wearable cardiac therapeutic device for improved skin comfort when worn by a patient is provided, the device comprising: at least one therapy electrode configured to deliver therapeutic electrical pulses to a patient's heart; and a support garment configured to support the at least one therapy electrode in electrical communication with the patient's body, the support garment comprising: at least one support pocket disposed on an inside surface of the support garment for supporting the at least one therapy electrode on the support garment; and a mesh interface formed as part of the at least one support pocket, the mesh interface configured to facilitate electrical contact between the at least one therapy electrode and the patient's skin, wherein the mesh interface comprises: a first side comprising hydrophobic dielectric fiber(s) proximate to one or more electrically conductive fluid deployment openings on a therapy electrode; a second side comprising hydrophilic dielectric fiber(s) proximate to the patient's skin, and conductive fiber(s) and/or conductive particles configured to be interspersed with the hydrophobic dielectric fiber(s) and with the hydrophilic dielectric fiber(s) such that the conductive fiber(s) and/or conductive particles conduct therapeutic electrical current from the therapy electrode to the patient's skin, wherein the mesh interface is configured to facilitate transfer of electrically conductive fluid from one or more electrically conductive fluid reservoirs disposed on the therapy electrode through the one or more electrically conductive fluid deployment openings of the therapy electrode and towards the patient's skin.

In some examples, a mesh interface for use with a support garment of a wearable cardiac therapeutic device is provided, the mesh interface comprising: a first side comprising hydrophobic fiber(s) proximate to one or more conductive fluid deployment openings on a therapy electrode; a second side comprising hydrophilic fiber(s) proximate to the patient's skin; and conductive fiber(s) and/or conductive particles configured to be interspersed with the hydrophobic fiber(s) and the hydrophilic fiber(s) such that the conductive fiber(s) and/or conductive particles conduct therapeutic electrical current from the therapy electrode to the patient's skin, wherein the mesh interface is configured to facilitate transfer of electrically conductive fluid from one or more electrically conductive fluid reservoirs disposed on the therapy electrode through the one or more electrically conductive fluid deployment openings of the therapy electrode and towards the patient's skin.

In some examples, a mesh interface for use with a support garment of a wearable cardiac therapeutic device is provided, the mesh interface comprising: a first side comprising fiber(s) proximate to one or more conductive fluid deployment openings on a therapy electrode; a second side comprising fiber(s) proximate to the patient's skin, wherein the fiber(s) of the second side are more hydrophilic than the fibers of the first side; and conductive fiber(s) and/or conductive particles configured to be interspersed with the fiber(s) of the first side and the fiber(s) of the second side such that the conductive fiber(s) and/or conductive particles conduct therapeutic electrical current from the therapy electrode to the patient's skin, wherein the mesh interface is configured to facilitate transfer of electrically conductive fluid from one or more electrically conductive fluid reservoirs disposed on the therapy electrode through the one or more electrically conductive fluid deployment openings of the therapy electrode and towards the patient's skin. The fiber(s) of the first side can comprise hydrophobic fibers.

In any of the examples of mesh interface(s), support garment(s) and/or wearable cardiac therapeutic device(s) described herein, the hydrophobic dielectric fiber(s) can be configured to facilitate movement of and/or pull the electrically conductive fluid from the one or more electrically conductive fluid deployment openings of the therapy electrode.

In any of the examples of mesh interface(s), support garment(s) and/or wearable cardiac therapeutic device(s) described herein, the hydrophilic dielectric fiber(s) can be configured to facilitate movement of and/or push the electrically conductive fluid towards the patient's skin.

In any of the examples of mesh interface(s), support garment(s) and/or wearable cardiac therapeutic device(s) described herein, the hydrophobic dielectric fiber(s) can be selected from the group consisting of polyester, polypropylene, olefin, acrylic, modacrylic, silk, hydrophobic nylon, wool, spandex, bamboo, and combinations thereof.

In any of the examples of mesh interface(s), support garment(s) and/or wearable cardiac therapeutic device(s) described herein, the hydrophobic dielectric fiber(s) can comprise fiber(s) treated to provide hydrophobicity.

In any of the examples of mesh interface(s), support garment(s) and/or wearable cardiac therapeutic device(s) described herein, the hydrophobic dielectric fiber(s) can comprise at least one hydrophobic coating and/or at least one hydrophobic impregnant.

In any of the examples of mesh interface(s), support garment(s) and/or wearable cardiac therapeutic device(s) described herein, the hydrophobic dielectric fiber(s) can comprise at least one hydrophobic coating and at least one hydrophobic impregnant which are chemically the same.

In any of the examples of mesh interface(s), support garment(s) and/or wearable cardiac therapeutic device(s) described herein, the hydrophobic dielectric fiber(s) can comprise at least one hydrophobic coating and at least one hydrophobic impregnant which are chemically different.

In any of the examples of mesh interface(s), support garment(s) and/or wearable cardiac therapeutic device(s) described herein, the hydrophilic dielectric fiber(s) are selected from the group consisting of cotton, wool, linen, acetate, cellulosic, rayon, hydrophilic nylon, polyester and combinations thereof.

In any of the examples of mesh interface(s), support garment(s) and/or wearable cardiac therapeutic device(s) described herein, the hydrophilic dielectric fiber(s) comprise fiber(s) treated to provide hydrophilicity.

In any of the examples of mesh interface(s), support garment(s) and/or wearable cardiac therapeutic device(s) described herein, the hydrophilic dielectric fiber(s) comprise at least one hydrophilic coating and/or at least one hydrophilic impregnate.

In any of the examples of mesh interface(s), support garment(s) and/or wearable cardiac therapeutic device(s) described herein, the hydrophilic dielectric fiber(s) comprise at least one hydrophilic coating and at least one hydrophilic impregnant which are chemically the same.

In any of the examples of mesh interface(s), support garment(s) and/or wearable cardiac therapeutic device(s) described herein, the hydrophilic dielectric fiber(s) comprise at least one hydrophilic coating and at least one hydrophilic impregnant which are chemically different.

In any of the examples of mesh interface(s), support garment(s) and/or wearable cardiac therapeutic device(s) described herein, the at least one of the hydrophobic dielectric fiber(s) and/or the hydrophilic dielectric fiber(s) comprise microfibers.

In any of the examples of mesh interface(s), support garment(s) and/or wearable cardiac therapeutic device(s) described herein, the conductive fiber(s) comprise silver-plated nylon yarn.

In any of the examples of mesh interface(s), support garment(s) and/or wearable cardiac therapeutic device(s) described herein, the conductive fiber(s) and/or conductive particles are configured to form a plurality of conductive pathways extending from the first side of the mesh interface to the second side of the mesh interface.

In any of the examples of mesh interface(s), support garment(s) and/or wearable cardiac therapeutic device(s) described herein, the mesh interface further comprises opening(s) extending through the mesh interface from the first side to the second side, the mesh interface being configured to facilitate transfer of the electrically conductive fluid from the at least one therapy electrode to the patient's skin via the opening(s).

In any of the examples of mesh interface(s), support garment(s) and/or wearable cardiac therapeutic device(s) described herein, at least a portion of the opening(s) are aligned with respective electrically conductive fluid deployment opening(s) on the therapy electrode to facilitate transfer of the electrically conductive fluid from the at least one therapy electrode to the patient's skin via the opening(s).

In any of the examples of mesh interface(s), support garment(s) and/or wearable cardiac therapeutic device(s) described herein, at least a portion of the opening(s) are formed by spaces between hydrophobic dielectric fiber(s), hydrophilic dielectric fiber(s), and/or conductive fiber(s).

In any of the examples of mesh interface(s), support garment(s) and/or wearable cardiac therapeutic device(s) described herein, each of the opening(s) independently has an average diameter of about 0.005 inches to about 0.3 inches (about 0.13 mm to about 7.6 mm).

In any of the examples of mesh interface(s), support garment(s) and/or wearable cardiac therapeutic device(s) described herein, the mesh interface is configured to receive electrically conductive fluid from the one or more electrically conductive fluid deployment openings of the at least one therapy electrode in an amount of about 0.1 cubic-centimeter (cc) to about 30 cc of electrically conductive fluid.

In any of the examples of mesh interface(s), support garment(s) and/or wearable cardiac therapeutic device(s) described herein, the electrically conductive fluid is configured to provide a predetermined electrical impendence of the plurality of conductive pathways extending through the mesh interface from the first side to the second side of about 0.01Ω to about 5Ω.

In any of the examples of mesh interface(s), support garment(s) and/or wearable cardiac therapeutic device(s) described herein, the mesh interface is further configured to be porous to the electrically conductive gel by a plurality of secondary openings within the mesh interface.

In any of the examples of mesh interface(s), support garment(s) and/or wearable cardiac therapeutic device(s) described herein, the second side of the mesh interface comprises the hydrophilic dielectric fiber(s) in a predetermined amount configured to provide a comfortable feel on the patient's skin.

In any of the examples of mesh interface(s), support garment(s) and/or wearable cardiac therapeutic device(s) described herein, the second side of the mesh interface comprises about 15 weight percent to about 85 weight percent of the hydrophilic dielectric fiber(s) on a basis of total weight of the hydrophilic dielectric fiber(s), and the conductive fiber(s), and the conductive particles (if present).

In any of the examples of mesh interface(s), support garment(s) and/or wearable cardiac therapeutic device(s) described herein, the second side of the mesh interface comprises about 25 weight percent to about 60 weight percent of hydrophilic dielectric fiber(s) on a basis of total weight of the hydrophilic dielectric fiber(s), and the conductive fiber(s), and the conductive particles (if present).

In any of the examples of mesh interface(s), support garment(s) and/or wearable cardiac therapeutic device(s) described herein, the second side of the mesh interface comprises about 30 weight percent to about 50 weight percent of hydrophilic dielectric fiber(s) on a basis of total weight of the hydrophilic dielectric fiber(s), and the conductive fiber(s), and the conductive particles (if present).

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the invention.

Further features and other examples and advantages will become apparent from the following detailed description made with reference to the drawings.

FIG. 1 is a schematic of an example wearable cardiac monitoring and therapeutic medical device that can be used in connection with the present disclosure;

FIG. 2 is a front view of an example support garment for the wearable cardiac monitoring and therapeutic medical device of FIG. 1 as worn on a patient;

FIG. 3 is a rear view of the support garment of FIG. 2 as worn on a patient;

FIGS. 4A and 4B are front views of an example support garment and electrode assembly, respectively, for a wearable monitoring and therapeutic medical device that can be used in connection with the present disclosure;

FIG. 5 is an enlarged front view of a mesh interface for a support pocket on the support garment of FIGS. 4A and 4B that can be used in connection with the present disclosure;

FIG. 6 is a further enlarged view of the mesh interface of FIG. 5 taken from area VI shown in FIG. 5;

FIG. 6A is an example of a mesh interface according to the view of FIG. 6;

FIG. 7 is a schematic perspective illustration of a portion of the mesh interface of FIG. 5;

FIG. 8 is a schematic cross-sectional illustration of the mesh interface taken along lines 8-8 shown in FIG. 7 as applied between a therapy electrode and a patient's skin;

FIGS. 9A and 9B are rear and front views, respectively, of a mannequin testing arrangement for measuring an impedance of the mesh interface according to an example of the present disclosure;

FIG. 10 is an enlarged front view of an example mesh interface

FIG. 11A is a schematic drawing showing a front perspective view of an example monitor for the wearable medical device of FIG. 4A;

FIG. 11B is a schematic drawing showing a rear perspective view of the example monitor of FIG. 11A;

FIG. 12 is a schematic diagram of functional components of the wearable medical device of FIG. 12;

FIG. 13 is a view of a portion of an example of a mesh interface;

FIG. 14 is a schematic view of a portion of an example of a mesh interface showing hydrophobic fibers, hydrophilic fibers and conductive particles and/or fibers;

FIG. 15 shows schematic representations of knitted mesh structures and woven mesh structures;

FIG. 16 is a schematic view of a portion of an example of a mesh interface showing hydrophobic fibers, less hydrophobic fibers and conductive particles and/or fibers;

FIG. 17 is a schematic drawing of a hook in weft knitting showing the positional relationship between strands of yarn;

FIG. 18 is a schematic drawing of plating in weft knitting;

FIG. 19 is a top plan view of a therapy electrode showing the gel reservoirs according to an example of the present disclosure;

FIG. 20 is a bottom plan view of the therapy electrode of FIG. 19 showing the bottom surface and exemplary electrically conductive fluid deployment openings therein according to an example of the present disclosure;

FIG. 21 is an example of an electrically conductive fluid or gel escape hole with a rupturable ring seal according to an example of the present disclosure; and

FIG. 22 is a front elevational view of the bottom surface of a therapy electrode showing disbursed conductive fluid after discharge from the reservoirs.

### DETAILED DESCRIPTION OF SOME OF THE EMBODIMENTS

As used herein, the singular forms of “a”, “an”, and “the” include plural referents unless the context clearly dictates otherwise.

As used herein, the terms “right”, “left”, “top”, and derivatives thereof shall relate to the invention as it is oriented in the drawing figures. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings and described in the following specification are examples. Hence, specific dimensions and other physical characteristics related to the embodiments disclosed herein are not to be considered as limiting.

For the purposes of this specification, unless otherwise indicated, all numbers expressing quantities of ingredients, reaction conditions, dimensions, physical characteristics, and so forth used in the specification and claims are to be understood as being modified in all instances by the term “about” or “about”. Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. Also, it should be understood that any numerical range recited herein is intended to include all sub-ranges subsumed therein. For example, a range of “1 to 10” is intended to include any and all sub-ranges between and including the recited minimum value of 1 and the recited maximum value of 10; that is, all subranges beginning with a minimum value equal to or greater than 1 and ending with a maximum value equal to or less than 10, and all subranges in between, e.g., 1 to 6.3, or 5.5 to 10, or 2.7 to 6.1.

As described herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data and does not actively transmit data to the second unit. As another example, a first unit can be in communication with a second unit if an intermediary unit processes data from one unit and transmits processed data to the second unit. It will be appreciated that numerous other arrangements are possible.

Patients who are at risk, have been hospitalized for, or otherwise are suffering from, adverse heart conditions can be prescribed a wearable cardiac monitoring and/or treatment device. Such a wearable device is generally prescribed for continuous or near-continuous use, e.g., only to be removed during initial set up, baselining, configuration updates under supervision of a trained caregiver or physician, when bathing, changing batteries, or changing garments. Accordingly, the patient wears the device during all daily activities such as walking, sitting, climbing stairs, resting or sleeping, and other similar daily activities. In this regard, maintaining continuous or near-continuous use of the device as prescribed can be important for monitoring patient progress as well as providing treatment to the patient if needed.

As summarized above, some examples disclosed herein are directed to a wearable cardiac therapeutic device, such as a non-invasive wearable ambulatory cardiac defibrillator, that improves therapy electrode contact with a patient's skin, device function, and patient comfort during wear. These wearable medical devices are used in clinical or outpatient settings to monitor and/or record various electrocardiogram (ECG) and other physiological signals of a patient. Moreover, these wearable medical devices can analyze the ECG and other physiological signals to monitor for arrhythmias, and, in example devices described herein, provide treatment such as cardioverting, defibrillating, or pacing shocks/pulses via therapy electrodes in the event of life-threatening arrhythmias. Such cardiac monitoring and treatment devices implement adjustable garment features and/or processes described herein, and includes wearable defibrillators, which are also called wearable cardioverter defibrillator (WCDs), and hospital wearable defibrillators (HWDs).

In some examples, a wearable cardiac therapeutic device comprises at least one therapy electrode configured to deliver therapeutic electrical pulses to a patient's heart. The device comprises a support garment configured to support the at least one therapy electrode in electrical communication with the patient's body. The support garment comprises at least one support pocket disposed on an inside surface of the support garment for supporting the at least one therapy electrode on the support garment. The support garment comprises a mesh interface formed as part of the at least one support pocket. The mesh interface is configured to facilitate electrical contact between the at least one therapy electrode and the patient's skin.

The therapy electrodes as described herein are configured to provide electrical treatment pulses/shocks delivered transcutaneously to the patient's heart via the mesh interface. A controller in communication with the therapy electrodes can control the timing and electrical properties of the cardioverting, defibrillating, and/or pacing shocks/pulses. In implementations, therapy electrodes as described herein comprise a base plate comprising a conductive surface configured to establish an electrical interface with the patient's skin.

Additionally or alternatively, the therapy electrodes described herein can also be configured to dispense electrically conductive fluid, for example an electrically conductive gel, onto the patient to improve the electrical interface between the conductive bottom surface of the electrodes and the skin. For example, each therapy electrode can include a plurality of openings on the conductive surface through which the electrically conductive fluid is dispersed onto the patient's skin. When the controller (as described in further detail below) determines that an electrical shock is warranted, the controller can cause the gel reservoirs to dispense the electrically conductive fluid in the interface between the conductive bottom surface and the patient's skin.

The electrically conductive fluid causes a decrease in electrical resistance between the conductive surface of the therapy electrode and the subject's skin. For example, the deployed electrically conductive fluid can help avoid causing burns on the subject's skin during therapy. Further, the deployed electrically conductive fluid can cause substantially all or most of the current from the therapeutic electrode components to be delivered to the subject's heart.

To effectively provide the treatment pulses described above, it is desirable that the therapy electrodes provided in the device are in appropriate electrical contact with the skin of the patient, and that the electrically conductive fluid ejected from the bottom surface of the therapy electrodes for delivery of the treatment pulses is rapidly deployed to the patient's skin in an appropriate manner.

In accordance with the disclosure herein, in one implementation, an example mesh interface is provided for use with a support garment of a wearable cardiac therapeutic device. The example mesh interface is configured to facilitate transfer of electrically conductive fluid from one or more electrically conductive fluid reservoirs disposed on the therapy electrode through the one or more electrically conductive fluid deployment openings of the therapy electrode and towards the patient's skin. The mesh interface in one example implementation comprises a first side comprising hydrophobic fiber(s) and/or hydrophobic dielectric fiber(s) proximate to one or more electrically conductive fluid deployment openings on a therapy electrode. The mesh interface comprises a second side comprising hydrophilic fiber(s) and/or hydrophilic dielectric fiber(s) proximate to the patient's skin. The mesh interface comprises conductive fiber(s) and/or conductive particles configured to be interspersed with the hydrophobic dielectric fiber(s) and with the hydrophilic dielectric fiber(s). The conductive fiber(s) and/or conductive particles are interspersed such that they conduct therapeutic electrical current from the therapy electrode to the patient's skin.

In another implementation, an example mesh interface comprises a first side comprising fiber(s) proximate to one or more conductive fluid deployment openings on a therapy electrode, and a second side comprising fiber(s) proximate to the patient's skin. In this implementation, the fiber(s) of the second side are more hydrophilic than the fibers of the first side. In such implementation, the conductive fiber(s) and/or conductive particles are configured to be interspersed with the fiber(s) of the first side and the fiber(s) of the second side such that the conductive fiber(s) and/or conductive particles conduct therapeutic electrical current from the therapy electrode to the patient's skin.

As described in further detail below, in some examples, this disclosure presents combinations of yarns to repel the conductive gel on an inside surface of the therapy electrode pocket (e.g., pushing the gel towards the patient's skin) and causes the gel to be disbursed on an outside surface of the therapy electrode pocket proximate the patient's skin without compromising long term on-patient comfort against the skin.

In such implementations, a hydrophobic yarn on the inside surface of the mesh interface and a hydrophilic yarn on the outside of the mesh interface can effectively set up a “push/pull” scenario whereby the gel would push through the inner pocket surface and spread to the exterior surface against the patient skin. As a further advantage of such constructions, the hydrophilic yarns proximate the patient's skin further helps with moisture management of the regular day to day use when the gel is not being disbursed.

In some examples, the yarns can be plated during the knitting process, for example to position hydrophobic yarns closer to the therapy electrode and/or to position hydrophilic yarns closer to the patient's skin.

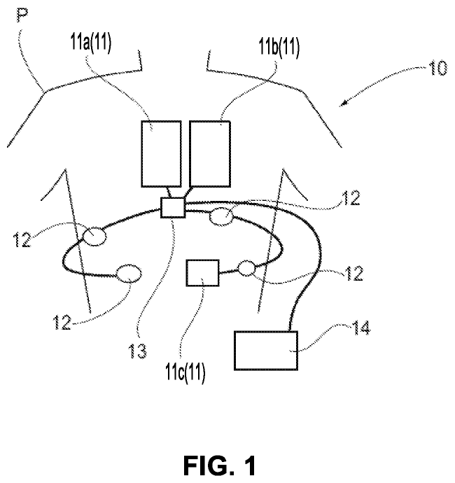

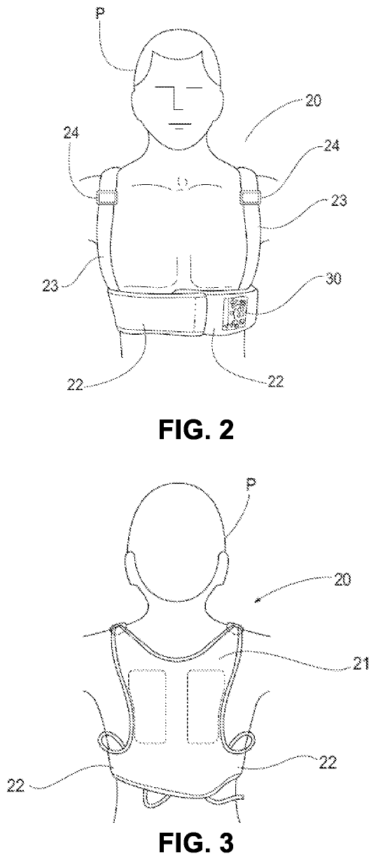

FIG. 1 illustrates an example of a wearable medical device that is external, ambulatory, and wearable by a patient P and is configured to implement one or more configurations described herein. For example, the wearable medical device can be an external or non-invasive medical device, e.g., the device configured to be located substantially external to the patient P. For example, the wearable medical device, shown in FIG. 1 as a wearable defibrillator 10, as described herein can be bodily-attached to the patient in the manner shown. The wearable defibrillator 10 can be worn about the torso of the ambulatory patient P. According to one example of the present disclosure, the wearable defibrillator 10 is used as an ambulatory cardiac monitoring and treatment device within a monitoring and treatment system according to the present disclosure. FIGS. 2-4B, 9A, 9B, 11A, 11B and 12, discussed in detail below, illustrate in further detail an example wearable medical device 10 implementing features, processes, and/or systems in accordance with the present disclosure. The wearable defibrillator 10 includes one or more therapy electrode(s) 11a-c (collectively 11) and a plurality of ECG monitoring electrodes 12. As shown in FIG. 1, the therapy electrodes 11 and ECG monitoring electrodes 12 are electrically coupled to a controller 14 via a plurality of cables or wires. In some examples, the wearable defibrillator 10 includes ECG processing and/or vibrational circuitry disposed within housing 13 as shown (described in further detail below). The circuitry in the housing 13 is electrically coupled to the controller 14 via at least one cable and/or wire.

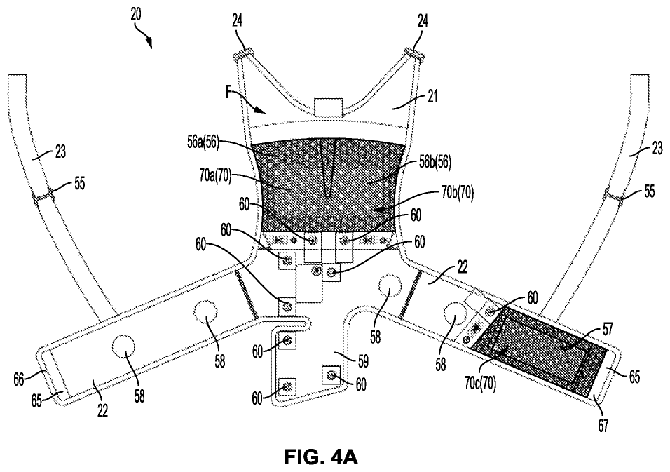

Referring now to FIGS. 2, 3 and 4B, in accordance with one or more examples, a support garment 20 is provided to keep the therapy electrodes 11 and sensing electrodes 12 of the wearable medical device in place against the patient's body while remaining comfortable during wear. FIGS. 2 and 3 illustrate such a support garment 20 in accordance with an example of the present disclosure.

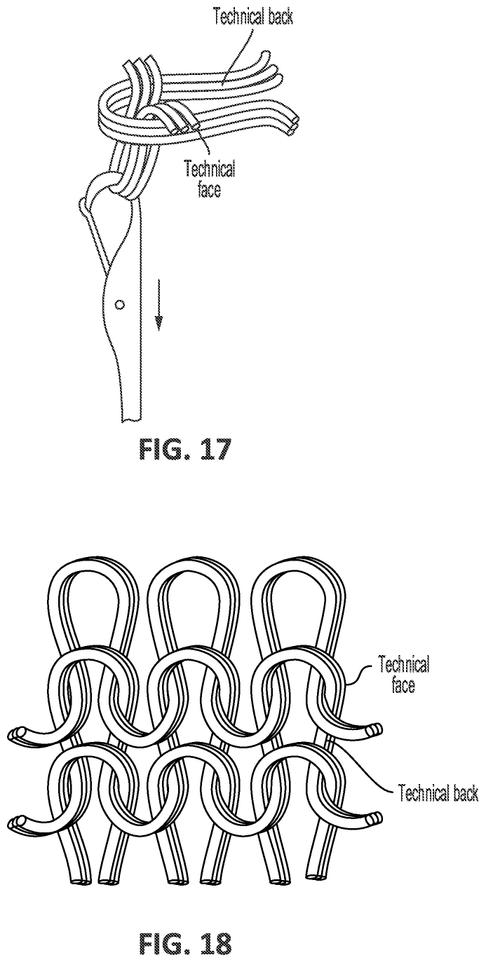

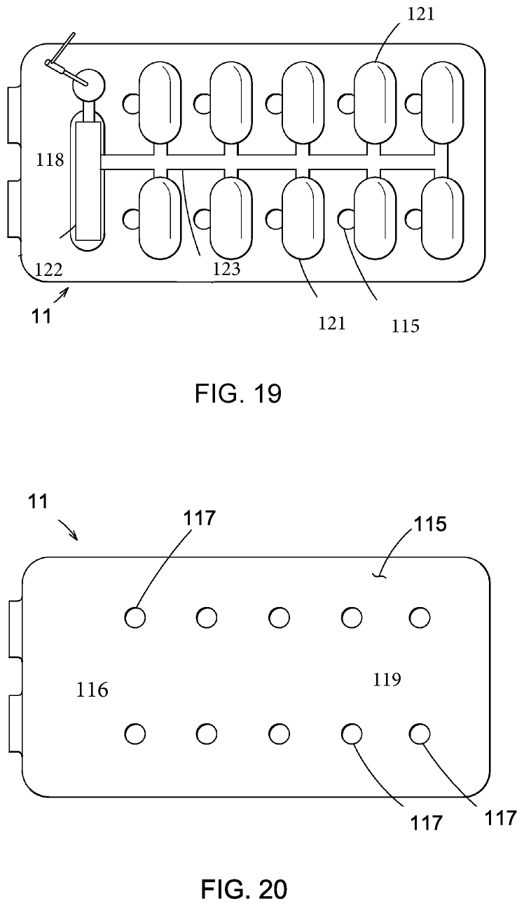

Referring briefly to FIGS. 19-22, in some examples, the therapeutic electrode component or therapy electrode 11 comprises a substrate or base plate 115 having a first side 118 (e.g., an upper side) and a second side 119 (e.g., a lower side) opposing the first side. As shown in FIG. 20, the base plate 115 comprises a conductive surface 116, such as stainless steel or other electrically conductive metallic material. In implementations, the conductive surface 116 is configured to be disposed within a therapy electrode pocket and in electrical contact with the patient's skin via a mesh interface as described herein, such that electrical therapy can be delivered to the subject through the conductive surface 116 and via the mesh interface.

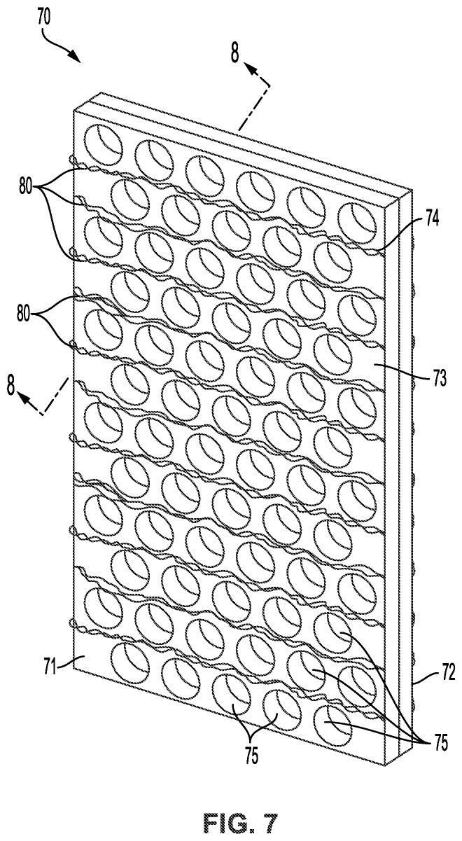

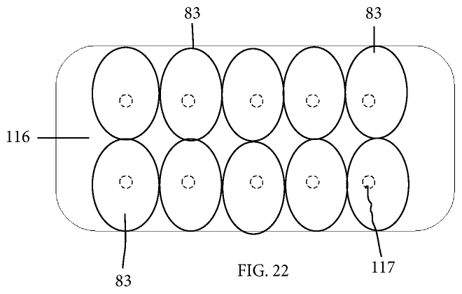

Referring now to FIG. 19, the first side 118 of the electrode base plate or substrate 115 comprises one or more repository(ies) or reservoir(s) 121 having an internal volume configured to releasably retain an electrically conductive fluid 83, for example a liquid or gel. FIG. 22 shows disbursement of the electrically conductive fluid 83, which is subsequently dispersed to the patient's skin via a mesh interface. In operation, the reservoir(s) 121 are caused to release the fluid 83 via fluid deployment openings 117. The electrically conductive fluid 83 is deployed onto the skin of the subject or patient and reduces the electrical impedance between the conductive surface 116 of the base plate 115 of the therapy electrode 11 and the skin of the patient P to facilitate the delivery of the electrical therapy to the subject via a mesh interface. For example, as shown in FIG. 8, fluid 83 can flow through openings 75 in the mesh interface 70.

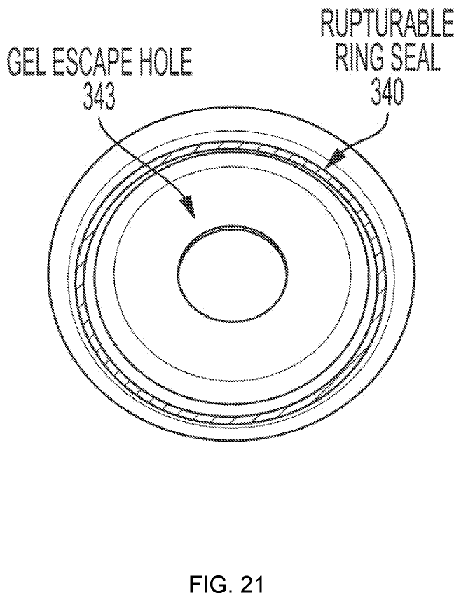

A rupturable membrane 340 or ring seal (FIG. 21) can be disposed between the internal volume of the repository and the conductive surface 116 of the base plate 115. When the rupturable membrane 340 is ruptured, the electrically conductive fluid 83 flows from the internal volume onto the conductive surface 116 via the fluid escape hole 343. The respective rupturable membranes 340 associated with each respective reservoir 121 are configured to rupture responsive to pressure being applied to the internal volumes of the reservoirs 121 so that the electrically conductive fluid 83 can flow out of the reservoirs 121 and onto the conductive surface 116 of the second side 119 of the base plate 115. Each reservoir 121 is associated with a fluid escape hole 343 to allow for the fluid to escape onto the conductive surface 116.

A fluid deployment device 122 (FIG. 19) can be configured to cause the reservoir 121 to release the electrically conductive fluid 83 onto the conductive surface 116 to reduce electrical impedance between the conductive surface 116 and skin of a subject. For example, conduit 123 is in fluid communication between the internal volumes of each reservoir 121 and the fluid deployment device, for example, a gas cartridge, air pump, or fluid pump also disposed on the base plate 115. The conduit 123 can comprise a central portion and branches leading to each respective reservoir 121. The conduit 123 provides for fluid to flow from the fluid deployment device 122 to the internal volumes of the reservoirs 121 to pressurize the internal volumes of the reservoirs 121 and cause the rupturable membrane 340 (shown in FIG. 21) to rupture and the electrically conductive fluid 83 to be pushed out of the reservoirs 121 through the ruptured rupturable membrane 340 via the fluid or gel escape holes 343, and out of the therapy electrode via the fluid deployment openings 117.

In some embodiments, the fluid deployment device 122 (FIG. 19) can include a pressurized working fluid source, e.g., a compressed non-noxious working fluid such as compressed nitrogen gas, compressed argon, etc., configured to supply pressurized working fluid to cause the reservoir to release the electrically conductive fluid 83. The fluid deployment device 122 can cause the reservoir 121 to release the electrically conductive fluid 83 onto the conductive surface to reduce electrical impedance between the conductive surface and the skin of the subject. The fluid deployment device 122 can be configured to provide sufficient force to expel the electrically conductive fluid 83 from the reservoir(s) 121 and out through the fluid deployment opening(s) 117 on the conductive surface 116 of the base plate 115 of the electrode 11, and into contact with the patient's skin. The fluid deployment device 122 can be configured to provide sufficient force to expel the electrically conductive fluid 83 from the reservoir(s) 121 and out through the fluid deployment opening(s) 117 on the conductive surface 116, through openings 75 or pores in the mesh interface 70, 570, and into contact with the patient's skin P (see FIG. 8).

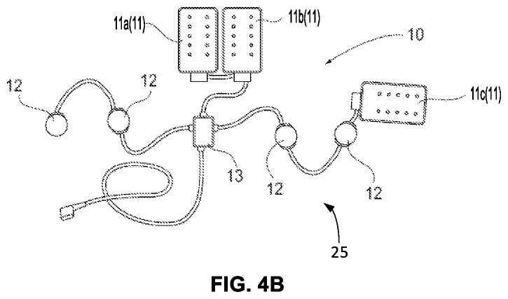

Referring now to FIG. 4A, the support garment 20 comprises rear support pockets 56a-b and a front support pocket 57 that incorporate mesh interfaces 70a-c (collectively 70). For example, the support garment 20 comprises rear support pockets 56 for removably receiving the posterior therapy electrodes 11a, b. The rear support pockets 56a, b incorporate rear pocket mesh interfaces 70a, b and is configured to position the posterior therapy electrodes 11a, b against a patient's posterior side (e.g., posterior upper torso region). The support garment 20 comprises a front support pocket 57 for removably receiving an anterior therapy electrode 11c. The front support pocket 57 incorporates mesh interface 70c and is configured to position the anterior therapy electrode against the patient's anterior side (e.g., anterior torso region on the patient's left side). The mesh interface 70 is configured to physically separate the conductive surface(s) 116 of the therapy electrode(s) 11 from the skin of the patient P while allowing for electrical contact between the therapy electrode(s) 11 and the patient's skin. In this manner, for example, the mesh interface 70 facilitates a comfortable feel on the patient's skin while continuing to maintain the electrical contact. The mesh interface 70 is configured to allow an electrically conductive fluid 83 that can be automatically ejected from a plurality of fluid deployment openings 117 on the conductive surface(s) 116 of the electrode(s) 11 to pass through the mesh interface 70 to disperse on the skin of the patient P. The mesh interface 70 is also configured to secure the therapy electrode(s) 11 within the support garment 20. In this manner, the mesh interface 70 ensures proper electrical contact, flow of electrically conductive fluid 83, and optimal comfort and feel against the patient's skin, even during body motions.

As also shown in FIGS. 4A and 4B, the support garment 20 can be provided in the form of a vest or harness having a back portion 21 and sides extending around the front of the patient to form a belt 22. The ends 66, 67 of the belt 22 are connected at the front of the patient by a closure mechanism 65. The support garment 20 can further comprise two straps 23 connecting the back portion 21 to the belt 22 at the front of the patient. The straps 23 have an adjustable size to provide a more customized fit to the patient. First strap slides 24 can be provided to connect the straps 23 to the back portion 21 of the support garment. Second strap slides 55 can be provided along the straps 23 to facilitate size adjustment of the straps 23. The straps 23 can also be selectively attached to the belt 22.

The support garment 20 can be configured for one-sided assembly of the electrode assembly 25 onto the support garment 20 such that the support garment 20 does not need to be flipped or turned over in order to properly position the therapy electrodes 11 and the sensing electrodes 12 on the support garment 20. The back portion 21 and the belt 22 of the support garment 20 can comprise attachment points 58 for supporting the sensing electrodes 12 in positions against the patient's skin in spaced locations around the circumference of the patient's chest. The attachment points 58 can include hook-and-loop fasteners for attaching ECG sensing electrodes 12 having a corresponding fastener disposed thereon to the inside surface of the belt 22. The attachment points 58 can be color coded to provide guidance for appropriately connecting the sensing electrodes 12 to the support garment 20. Additionally or alternatively, one or more of the ECG sensing electrodes can be permanently integrated into the belt 22 of the support garment 20, e.g., such that they cannot be removed/replaced by a patient during use. The support garment 20 can further comprise a flap 59 extending from the back portion 21. The flap 59 and the back portion 21 include fasteners 60 for connecting the flap 59 to the inside surface of the back portion 21 in order to define a pouch or pocket for holding a ECG signal processing and/or vibrational circuitry disposed in housing 13 of the electrode assembly 25. For example, the processing and/or vibrational circuitry unit 13 can include ECG acquisition and conditioning circuitry configured to receive ECG signals from the plurality of ECG sensing electrodes 12, amplify the signals, condition (e.g., using filter circuits) to remove noise, and sample the signal to produce a digitized ECG signal corresponding to the analog ECG input. In examples, the unit 13 can also comprise vibrational circuitry configured to receive an input from a controller (e.g., controller 14 shown in FIG. 1) and provide the patient a vibrational alarm or notification as appropriate. The outer surface of the belt 22 can further comprise a schematic 30 (shown in FIG. 2) imprinted on the fabric for assisting the patient or medical professional in assembling the electrode assembly 25 onto the support garment 20.

In examples, the mesh interface 70 facilitates monitoring of the electrical contact between the therapy electrode(s) 11 and the patient's skin such that a warning can be provided by the wearable defibrillator controller 14 (shown in FIG. 1) if the therapy electrode(s) 11 is not making appropriate electrical contact with the patient's skin. Another pocket, front pocket 57, including a mesh interface 70c according to a similar construction, is included on an inside surface of the belt 22 for receiving a front therapy electrode 11c (shown in FIG. 4B) and holding the electrode 11c in position against the patient's anterior side. Briefly referring to FIG. 4B, there is shown the components of an electrode belt 25 configured to be disposed on the support garment 20. The electrode belt 25, as shown, includes one or more therapy electrode(s) 11a-c (collectively 11) and a plurality of ECG monitoring electrodes 12. The therapy electrodes 11 and ECG monitoring electrodes 12 are electrically coupled to a controller 14 (FIG. 1) via a connector. The wearable defibrillator 10 includes ECG processing and/or vibrational circuitry disposed within housing 13 as shown and coupled to the various therapy electrodes 11 and ECG monitoring electrodes 12 as shown.

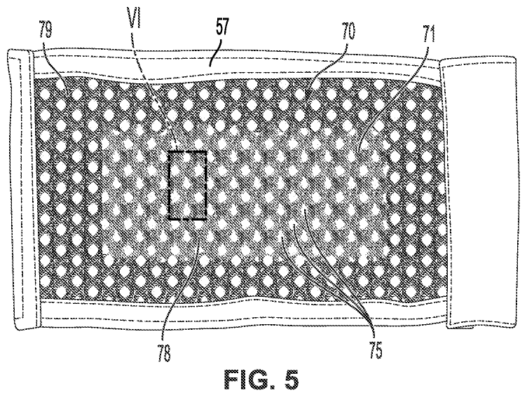

After assembly of the therapy electrode(s) 11 into the respective pocket(s) 56, 57, the pocket(s) 56, 57 are closed on the support garment 20, by a fastener or fasteners 60, such as a button or snap. Further details regarding the mesh interfaces 70a, 70b, 70c of the pockets 56, 57 will be discussed in detail below with reference to FIGS. 5-8, 10, and 13-16. In FIG. 4A, two rear pockets 56a and 56b, and one front pocket 57 are shown corresponding to the two rear therapy electrodes 11a and 11b, and front therapy electrode 11c, respectively. In other implementations, fewer or more rear or front pockets and/or therapy electrodes can be provided. For example, a garment can include two rear pockets and two front pockets, these pockets configured to receive two rear therapy electrodes and two front therapy electrodes. For example, a garment can include three rear pockets and three front pockets, these pockets configured to receive three rear therapy electrodes and three front therapy electrodes. In such examples, the rear or front pockets can include corresponding mesh interface(s) as described herein.

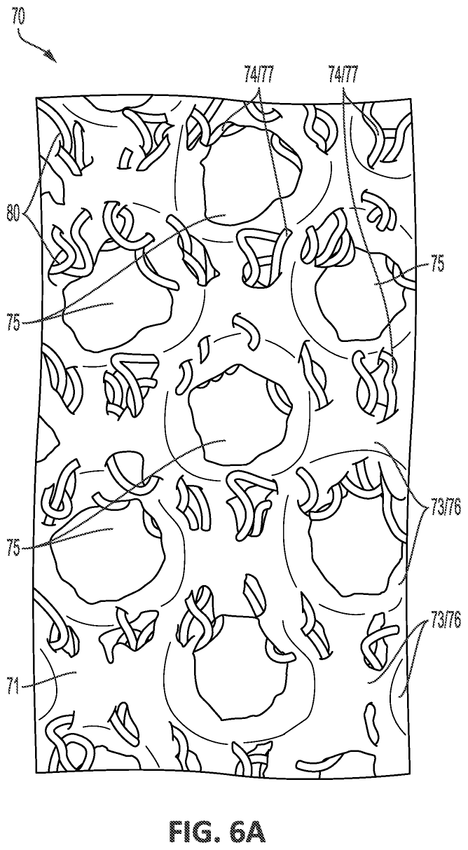

Referring now to FIGS. 7 and 8, the mesh interface 70 comprises a first side 71 oriented generally towards the conductive surface 116 of at least one therapy electrode 11 and a second side 72 oriented generally towards the patient's skin.

The mesh interface 70 is configured to facilitate transfer of electrically conductive fluid 83 from one or more electrically conductive fluid reservoirs 121 disposed on the therapy electrode 11 through the one or more electrically conductive fluid deployment openings 117 of the therapy electrode 11 and towards the patient's skin.

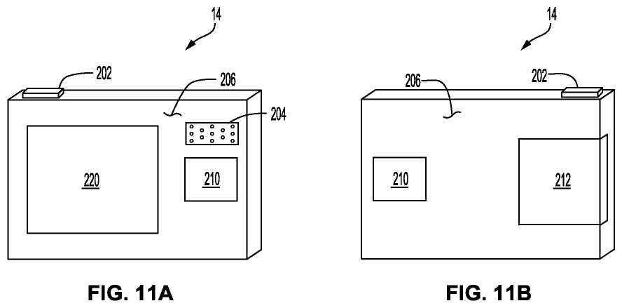

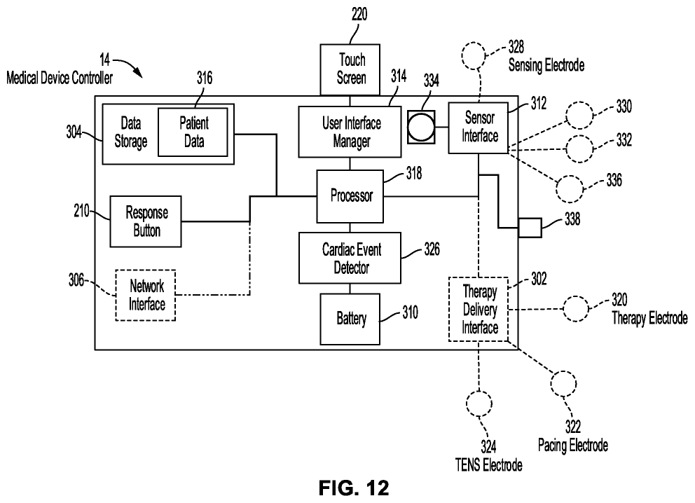

In some examples, as shown schematically in FIGS. 13 and 14, the mesh interface 70 comprises a first side 71 comprising one or more hydrophobic dielectric fiber(s) and/or hydrophobic fiber(s) 73a (referred to collectively throughout as “hydrophobic fiber(s)”, unless indicated otherwise) proximate to one or more electrically conductive fluid deployment openings 117 on a therapy electrode 11. The mesh interface 70 comprises a second side 72 comprising one or more hydrophilic dielectric fiber(s) and/or hydrophilic fiber(s) 73b (referred to collectively throughout as “hydrophilic fiber(s)”, unless indicated otherwise) proximate to the patient's skin. One or more conductive fiber(s) and/or particles 74 are configured to be interspersed with the hydrophobic dielectric fiber(s) and/or hydrophobic fiber(s) 73a and with the hydrophilic dielectric fiber(s) and/or hydrophilic fiber(s) 73b, such that the conductive fiber(s) and/or conductive particle(s) 74 form conductive pathways 80 (e.g., shown schematically in FIG. 8) to conduct therapeutic electrical current from the therapy electrode 11 to the patient's skin.

The mesh interface 70 is further constructed to facilitate transfer of electrically conductive fluid 83 from one or more electrically conductive fluid reservoirs 121 disposed on the therapy electrode 11 through the one or more electrically conductive fluid deployment openings 117 of the therapy electrode 11 and towards the patient's skin. The electrically conductive fluid 83 facilitates the conduction of the therapeutic electrical current from the therapy electrode 11 to the patient's skin via the conductive pathways, e.g., by lowering the electrical impedance between the conductive surface of the therapy electrode 11 and the patient's skin.

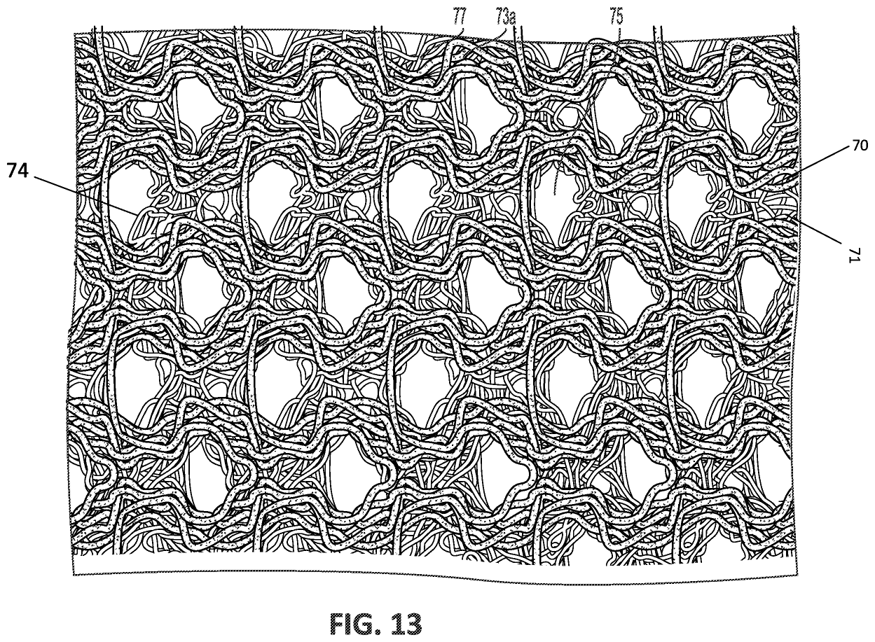

Referring now to FIG. 15, there is shown example schematic representations of a knitted mesh structure 70′ and a woven mesh structure 70″. In these representations, mesh structures 70 with uncoated fibers on first and second sides are compared to mesh structures 70 with hydrophobic coated fibers on the first side and hydrophilic coated fibers on the second side. As shown, in both knitted and woven implementations of the mesh structures 70, a drop of water is further absorbed through the thickness of the mesh structure 70 with coated fibers than the mesh structure 70 with uncoated fibers. In this manner, FIG. 15 illustrates example benefits of providing hydrophobic and hydrophilic fibers on opposing sides of mesh interfaces 70.

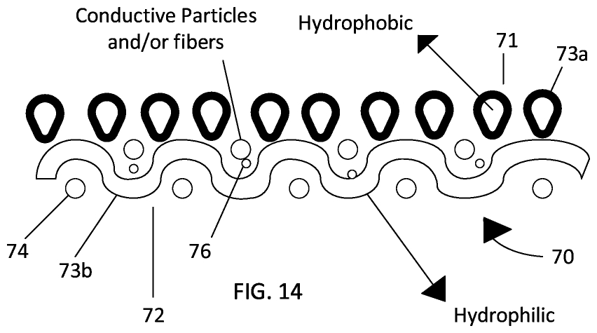

In some examples, as shown for example in FIG. 16, the mesh interface 570 comprises a first side 571 comprising one or more fiber(s) 573(a) proximate to one or more conductive fluid deployment openings 117 on a therapy electrode 11. The mesh interface 570 also comprises a second side 572 comprising one or more fiber(s) 573(b) proximate to the patient's skin, wherein the fiber(s) 573(b) of the second side 572 are more hydrophilic than the fibers 573(a) of the first side 571. The mesh interface 570 comprises one or more conductive fiber(s) and/or conductive particles 574 which are configured to be interspersed with the fiber(s) 573(a) of the first side 571 and the fiber(s) 573(b) of the second side 572 such that the conductive fiber(s) and/or conductive particles 574 form conductive pathways 580 to conduct therapeutic electrical current from the therapy electrode to the patient's skin.

The mesh interface 570 is further constructed to facilitate transfer of electrically conductive fluid 83 from one or more electrically conductive fluid reservoirs 121 disposed on the therapy electrode 11 through the one or more electrically conductive fluid deployment openings 117 of the therapy electrode 11 and towards the patient's skin. The electrically conductive fluid 83 facilitates the conduction of the therapeutic electrical current from the therapy electrode 11 to the patient's skin via the conductive pathways, e.g., by lowering the electrical impedance between the conductive surface of the therapy electrode 11 and the patient's skin. In some of these examples, some or all of the fiber(s) 573(b) of the second side 572 are more hydrophilic than some or all of the fibers 573(a) of the first side 571. For example, some or all of the fibers 573(a) of the first side 571 can be hydrophobic and some or all of the fiber(s) 573(b) of the second side 572 can be hydrophilic to facilitate transfer of electrically conductive fluid 83 from one or more electrically conductive fluid reservoirs 121 disposed on the therapy electrode 11 through the one or more electrically conductive fluid deployment openings 117 of the therapy electrode 11 and towards the patient's skin. In other examples, some or all of the fibers 573(a) of the first side 571 can be hydrophilic and some or all of the fiber(s) 573(b) of the second side 572 can be more hydrophilic than some or all of the fibers 573(a) of the first side 571 to facilitate transfer of electrically conductive fluid 83 from one or more electrically conductive fluid reservoirs 121 disposed on the therapy electrode 11 through the one or more electrically conductive fluid deployment openings 117 of the therapy electrode 11 and towards the patient's skin.

While not intending to be bound by any theory, the use of hydrophobic fiber(s) proximate to one or more electrically conductive fluid deployment openings 117 on the therapy electrode 11, and less hydrophobic (as compared to the hydrophobic fiber(s) proximate to openings 117) and/or hydrophilic fiber(s) proximate to the patient's skin, can enhance the movement of water-based electrically conductive fluid 83 expelled from the fluid deployment openings 117 towards the patient's skin by wicking and/or capillary action through the mesh interface. As described herein, hydrophobic materials or fibers tend to repel water, lack an affinity for water, tend not to appreciably absorb water, and/or tend not to dissolve in water. Thus, the water-based electrically conductive fluid 83 is not attracted to the hydrophobic fiber(s) closer to the fluid deployment openings 117 of the electrode 11. However, the water-based electrically conductive fluid 83 is attracted to the more hydrophilic fiber(s) (e.g., less hydrophobic fiber(s) and/or hydrophilic fiber(s)) in the mesh interface closer to the patient's skin. As described herein, hydrophilic materials or fibers have an affinity for water, tend to attract water, tend to absorb water, and/or tend to dissolve in water.

Openings 75, 575 in the mesh interface (e.g., shown in FIGS. 5-8 and 16) provide relatively larger conduits to permit passage of the electrically conductive fluid 83 from the electrically conductive fluid deployment openings 117 to the patient's skin, while the mesh interface structure helps to maintain the therapy electrode 11 in the proper position and provides more comfortable wearability of the support garment for the patient. The spaces between the fiber(s) and/or yarn(s) of the mesh interface 70, 570 provide capillary pathways to transport the electrically conductive fluid 83 between the first side 71, 571 and the second side 72, 572 of the mesh interface 70, 570.

In examples, capillary action within the textile fabric can be characterized based on the diameter of capillary tube(s) within the textile fabric material. Additionally or alternatively, in some examples capillary action may be characterized by surface tension of the conductive fluid 83 in contact with the surfaces of the capillary tube(s) (e.g., adhesion forces to the surfaces of the textile material being greater than cohesive forces between molecules of the conductive fluid 83). In this regard, a smaller diameter and/or greater surface tension increases the tendency for the conductive fluid 83 to move through the capillary pathways in the textile fabric material. Hydrophilic fibers have a higher surface tension and as such more readily attract or absorb moisture compared to hydrophobic fibers. Fibers with lower absorbency, such as hydrophobic fiber(s), can wick moisture rapidly through a fabric compared to fibers with greater absorbency, such as hydrophilic fiber(s). The lack of affinity or repelling effect of moisture by the hydrophobic fibers(s) combined with a greater affinity and attraction for moisture by the hydrophilic fiber(s) can lead to a “push/pull” effect, which enhances the movement of the electrically conductive fluid 83 from the region of the first side 71, 571 proximate to the fluid deployment openings 117 having more hydrophobic fibers towards the region of the second side 72, 572 proximate to the skin having more hydrophilic fibers (or less hydrophobic compared to the hydrophobic fibers of the first side).

In some examples, the transition from more hydrophobic fibers proximate the first side to more hydrophilic fibers can be gradual across the thickness of the mesh interface between the first side and the second side, or designed in two or more stages across the thickness of the mesh interface. For example, the weight percentage of hydrophobic fiber(s) proximate to the first side 71, 571 of the mesh interface 70, 570 can range from about 70 to 85 weight percent proximate to the openings 117, about 45 to about 60 weight percent proximate to the midpoint of the thickness of the mesh interface, and/or about 5 to about 25 weight percent proximate to the second side of the mesh interface (proximate to the skin), based upon total weight of the hydrophobic fiber(s), hydrophilic fiber(s) and conductive fibers or particles of the mesh interface. For example, the weight percentage of hydrophilic fiber(s) proximate to the second side 72, 572 of the mesh interface 70, 570 can range from about 70 to 85 weight percent proximate to the skin, about 45 to about 60 weight percent proximate to the midpoint of the thickness of the mesh interface, and/or about 5 to about 25 weight percent proximate to the first side of the mesh interface (proximate to the openings 117, based upon total weight of the hydrophobic fiber(s), hydrophilic fiber(s) and conductive fibers or particles of the mesh interface. In examples, the weight percentage of conductive fibers or particles in the mesh structure can range from about 1 to about 2.5 percent, about 2.5 to about 5 percent, about 5 to about 10, about 10 to about 25, or about 25 to about 50, or about 50 to about 75, based upon total weight of the hydrophobic fiber(s), hydrophilic fiber(s), and conductive fibers or particles of the mesh interface. The weight percentage can be determined, for example, by weighing the weight percent of hydrophobic fiber(s) or hydrophilic fiber(s) in a fabric sample measuring two inches×two inches in a plane generally parallel to the nearest side, where the sample is in a relaxed, uncompressed and unstretched state.

Examples of electrically conductive fluids, for example liquids or gels, for use in connection with embodiments are described below. Some examples of electrically conductive fluids include SignaGel Electrode Gel from Parker Laboratories, Inc. of Fairfield, N.J., USA, Quick Recovery (Q.R.) defibrillator gel from Kettering Surgical Appliances Limited of Northampton, United Kingdom, or Quick Recovery defibrillator gel from Pharmaceutical Innovations, Inc. of Newark, N.J., USA.

In some examples, the electrically conductive fluid 83 can have a viscosity of about 2,000 centipoise (cps) to about 70,000 cps, or about 10,000 to about 50,000 cps, or about 20,000 to about 45,000 cps, or about 33,000 to about 40,000 cps. The viscosity can be determined using a standard Viscometer, such as a Brookfield Synchro-Lectric Viscometer, Model LVT or equivalent. The fluid 83 sample is prepared as follows. A 600 mL low form Griffin beaker is used, and filled past the 400 mL mark. The test sample is brought to 25° C., and the temperature is periodically checked maintaining to about +/−0.2 C. With respect to the test procedure, initially, the leg guard is attached to the viscometer. Then the spindle is attached to the viscometer by holding the viscometer connector steady with an upward force while screwing the spindle on in a left hand thread. The power to the viscometer is turned on and set the following: Speed—6.0 RPM; Spindle Code—#5. The beaker is centered under the leg guard, and immerse the spindle so that the notch in spindle is at the liquid level. Care should be taken to avoid entrapping air beneath the spindle. Then, the spindle rotation is engaged and viscosity measurement is made between 30 seconds to 60 seconds.

The electrical resistance of the electrically conductive fluid 83 itself can range from about 0.01Ω to about 50Ω, or about 3Ω to about 20Ω, or about 5Ω to about 15Ω. The resistance can be determined using a standard A.C. bridge circuit, such as a Belco A.C. Bridge, Model BR-8, using two half-inch spherical electrodes located at about 1-2 inches apart and immersed within the conductive gel at a depth of 1 inch+/−0.032″, at a temperature of about 25° C. In the test, the voltage can be set to about IV, sine wave at 1 kHz, with a 0.1 divider. A B&K Precision 3011B 2 MHz function generator or equivalent can be used to generate the test signal. A Fluke 45 Dual display multimeter or equivalent can be used to read the voltage in mV or Volts. The fluid 83 can be prepared as follows. A 600 mL low form Griffin beaker is used, and filled past the 400 mL mark. The test sample is brought to 25 C, and the temperature is periodically checked maintaining to about +/−0.2 C.

In some examples, the electrically conductive fluid 83 is configured to provide a predetermined electrical impendence of the plurality of conductive pathways extending through the mesh interface 70, 570 from the first side 71, 571 to the second side 72, 572 of about 0.01Ω to about 10Ω, or about 0.01Ω to about 5Ω, or about 0.1Ω to about 2Ω, or about 0.25Ω to about 1.5Ω. The electrical impedance of the electrically conductive fluid 83 can be determined using the “mannequin test” described below. It is to be appreciated that the electrically conductive fluid 83 can be configured to provide a predetermined electrical impedance of the plurality of conductive pathways of suitable value.

Generally, the plurality of conductive fibers and/or conductive particles 74, 574 are interspersed and/or intertwined within and/or between the hydrophobic fiber(s) 73(a), 573(a) and within and/or between the hydrophilic fiber(s) 73(b), 573(b) to form a plurality of conductive pathways 80, 580 (e.g., schematically shown in FIG. 8) extending through the mesh interface 70, 570 from the first side 71, 571 to the second side 72, 572. The plurality of conductive pathways 80, 580 are configured to conduct the therapeutic electrical pulses through the mesh interface 70, 570 from the at least one therapy electrode 11 to the patient P.

According to some examples, the plurality of conductive fibers and/or conductive particles 74, 574 are interspersed within the mesh interface 70, 570 in a concentrated manner at distinct locations throughout at least a portion of the mesh interface 70, 570 to form sufficient conductive pathways 80, 580 extending through the mesh interface 70, 570 from the first side 71, 571 to the second side 72, 572. As described below, the mesh interface 70, 570 is configured to comprise conductive pathways 80, 580 distributed such that the mesh interface 70, 570 has a sufficiently low impedance to allow for the therapeutic electrical pulses to be conducted from the at least one therapeutic electrode 11 to the patient's skin P. According to the example, the amount of conductive metallic material incorporated into the mesh interface 70, 570 is reduced in comparison to a mesh fabric coated entirely with conductive metallic material. According to the example, the reduced amount of conductive metallic material incorporated in the mesh interface 70, 570 allows for the inclusion of the hydrophobic fiber(s) 73(a), 573(a) and the hydrophilic fiber(s) 73(b), 573(b) into the mesh interface 70, 570.

In some examples, the conductive pathways 80, 580 project from the first side 71, 571 and the second side 72, 572 of the mesh interface 70, 570, for example as shown in FIGS. 7 and 8, so as to be able to establish electrical contact with the therapy electrode 11 and the patient's skin P, respectively. In some examples, the mesh interface 70, 570 can also be configured to transmit one or more electrical signals (e.g., therapy electrode “fall off” signal) from the at least one therapy electrode 11 to the patient's skin. For example, the therapy electrode “fall off” signal can be used by the controller 14 to determine that the at least one therapy electrode 11 is present and/or correctly positioned on the patient's body.

In examples, fiber(s) can be present as individual strands of fiber(s) or yarn, in yarn(s) comprising multiple strands, and/or in thread(s) comprising multiple yarns plied together. In some examples, the yarn(s) can comprise two or more strands, for example three or more strands, four or more strands, five or more strands, etc. In some examples, the thread(s) comprise a single ply or two or more plies of yarn, each ply comprising one or more, or two or more, strands twisted together. For example, the linear mass density of the yarn can range from about 50 to about 1000 denier by 2 ply. In some examples, the yarn can be 3 ply, 4 ply or 5 ply.

In some examples, suitable hydrophobic fibers can be formed from hydrophobic materials, at least partially or fully coated with a hydrophobic material to provide hydrophobicity to a fiber, and/or impregnated with a hydrophobic material to provide hydrophobicity to a fiber.

The hydrophobicity of fiber(s) or yarns can be determined by knitting or weaving a sample of a solid knit fabric from the fiber(s)/yarn(s) used to prepare the first side of the mesh interface. For example, the sample could be knitted in the form of a Single Jersey knit structure. For example, the sample could be knitted in the form of a Pointelle structure, Flat or Jersey Knit structure, Purl Knit structure, Rib Stitch Knit structure, Interlock Stitch Knit structure, Double Knit structure, Warp Knitted structure, Tricot Knit structure, or Raschel Knit structure.

To determine relative hydrophobicity of two different fiber/yarn configurations for the first side, a second sample can be knitted or woven from the second set of fiber(s)/yarn(s) for the first side in the same manner as for the first sample, and tested to compare the relative hydrophobicity between the two samples. The hydrophobicity of the fabric can be determined using an electrically conductive fluid 83 such as are disclosed herein, according to AATCC (American Association of Textile Chemists and Colorists) Test Method: TM22-2017e (2019), incorporated by reference herein.

In some examples, the hydrophobic fiber(s) can be formed from materials comprising one or more of polyester, polypropylene, polytetrafluoroethylene (PTFE), olefin, acrylic, modacrylic, silk, hydrophobic nylon, wool, spandex, bamboo, and combinations thereof. In some examples, the foregoing materials may be treated to transform and/or enhance hydrophobicity properties for use in the implementations described herein. In some examples, the materials may be naturally hydrophobic and as such no or minimal such treatment may be needed. Example treatment techniques are referenced below.

Non-limiting examples of suitable hydrophobic polyester fibers comprise poly(ethylene terephthalate) (PET) fibers, and RESIST₂O PFOA free C₆ fluorocarbon treated fibers from Unifi Manufacturing, Inc. of Greensboro, N.C. Non-limiting examples of suitable polypropylene fibers comprise STANTEX® polypropylene from Pulcra Chemicals of Munich, Germany. Non-limiting examples of suitable olefinic fibers comprise polypropylene fibers and polyethylene fibers. Non-limiting examples of suitable acrylic fibers comprise ACRILAN. CRESLAN, ORLON, SAYELLE, and ZEFRAN acrylic fibers. Non-limiting examples of suitable modacrylic fibers comprise ELURA, SEF, VEREL, and ZEFRAN modacrylic fibers. Non-limiting examples of wool based hydrophobic fiber(s) comprise Optim™ water resistant wool from the Woolmark Company, Australia. For example, such wool based hydrophobic fiber(s) are prepared from Merino wool fibres that are pre-stretched and spun into yarn before being woven and/or knitted at very high levels of thread density in warp and weft. When the fabric is wet-finished, the stretch is released causing the yarns to contract, thus leading to a tightening of the fabric structure and the creation of a dense fabric structure. Non-limiting examples of suitable hydrophobic nylon fibers comprise hydrophobic treated Nylon 6, Nylon 6,6, Nylon 4,6, Nylon 6,9, Nylon 6,10, Nylon 6,12, Nylon 11, and Nylon 12. Non-limiting examples of suitable spandex fibers comprise LYCRA® spandex (available from the Lycra Company), such as LYCRA 25® spandex or ROICA polyether-type spandex, elastane, and ELASPAN (available from Invista of Witchita, Kans.).

In some examples, the hydrophobic fiber(s) comprise fiber(s) treated to provide hydrophobicity, such as treated nylon fibers. In some examples, the hydrophobic dielectric fiber(s) can comprise at least one hydrophobic coating and/or at least one hydrophobic impregnant. In some examples, the hydrophobic fiber(s) can comprise at least one hydrophobic coating and at least one hydrophobic impregnant which are chemically the same, e.g., have the same chemical components in the same amounts. In some examples, the hydrophobic fiber(s) comprise at least one hydrophobic coating and at least one hydrophobic impregnant which are chemically different, e.g., have the same chemical components in different amounts or have one or more chemically different components, for example different chemical structure or different molecular weights.

In some examples, the fibers can be impregnated or coated when the fibers are being drawn or spun into yarns, or impregnated or coated by application as a finish after drawing or spinning. Examples of hydrophobic coating and/or impregnant treatments include chemical treatments such as alkaline hydrolysis for polyesters, enzymatic treatment such as lipase treatment for polyesters, plasma or argon glow discharge treatment, and hydrophobic amino silicone treatments.

In some examples, a hydrophobic coating such as ECOREPEL® coating (commercially available from Schoeller of Switzerland) can be applied to the fiber(s). ECOREPEL® hydrophobic coating is based on long paraffin chains that wrap themselves around fibers to reduce the surface tension so that water droplets run off, and the feel of the fabric remains soft. Another example of a suitable coating is NANOSPHERE® coating which is commercially available from Schoeller of Switzerland.

In some examples, the second side 72, 572 comprises hydrophilic fiber(s) and/or hydrophilic dielectric fiber(s) 73(b), 573(b) proximate to the patient's skin. In some examples the fiber(s) of the second side are more hydrophilic than the fibers of the first side. The more hydrophilic fiber(s) are configured to facilitate movement of and/or push the electrically conductive fluid 83 towards the patient's skin.

In some examples, suitable hydrophilic fibers can be formed from hydrophilic materials, at least partially or fully coated with a hydrophilic material to provide hydrophilicity to the fiber, and/or impregnated with a hydrophilic material to provide hydrophilicity to the fiber.

The hydrophilicity of fiber(s) or yarns can be determined by knitting or weaving a sample of a solid knit fabric from the fiber(s)/yarn(s) used to prepare the second side of the mesh interface. If desired, to determine relative hydrophilicity of two different fiber/yarn configurations for the second side, a second sample can be knit or woven from the second set of fiber(s)/yarn(s) for the second side in the same manner as for the first sample, and tested to compare the relative hydrophilicity between the two samples. The hydrophilicity of the fabric can be determined using an electrically conductive fluid 83 such as are disclosed herein, using AATCC Test Method: TM197-2011e2(2018)e, incorporated by reference herein.

In some examples, the hydrophilic fibers can be formed from materials comprising one or more of cotton, wool, linen, acetate, cellulosic, rayon, hydrophilic nylon, polyester and combinations thereof. Non-limiting examples of suitable acetate fibers comprise diacetate, triacetate, CELAIRE available from Celanese, CHROMSPUN available from Eastman Chemical, ESTRON available from Eastman Chemical, SOALON available from Mitsubishi Chemical, LYNDA available from Mitsubishi Chemical, TENITE available from Eastman Chemical, ARNEL available from Celanese, TRICEL, ACELE, and AVISCO. Non-limiting examples of suitable hydrophilic nylon fibers comprise hydrophilic Nylon 6, Nylon 6.6 such as Sensil® Aquarius nylon 6.6 fiber fabric available from NILIT. Nylon 4,6. Nylon 6,9, Nylon 6,10, Nylon 6,12, Nylon 11, and Nylon 12, and TOPLON Nylon yarns available from Hyosung. Non-limiting examples of suitable polyester fibers comprise SORBTEK® recycled polyester available from Unifi, and COOLMAX polyester fibers from The Lycra Company.

In some examples, the hydrophilic fiber(s) comprise fiber(s) treated to provide hydrophilicity. In some examples, the hydrophilic fiber(s) can comprise at least one hydrophilic coating and/or at least one hydrophilic impregnant. In some examples, the hydrophilic fiber(s) can comprise at least one hydrophilic coating and at least one hydrophilic impregnant which are chemically the same, e.g., have the same chemical components in the same amounts. In some examples, the hydrophilic fiber(s) comprise at least one hydrophilic coating and at least one hydrophilic impregnant which are chemically different, e.g., have the same chemical components in different amounts or have one or more chemically different components, for example different chemical structure or different molecular weights.

In some examples, the fibers can be impregnated or coated when the fibers are being drawn or spun into yarns, or impregnated or coated by application as a finish after drawing or spinning. For example, cotton fibers can be made hydrophobic using a durable water repellant (DWR) finish. For example, DWR finish can be implemented as a coating added to the fiber to help fluids bead up and roll off the material. For example, a fluorinated DWR finish includes a functional coating containing perfluorocarbons. In implementation, a DWR treatment transforms cotton or other dielectric fibers into hydrophobic fibers. For example, a DWR process may create microscopic spikes on a surface which increases surface tension when fluid comes in contact with the surface and causes the fluid droplets to band together at the outer edge the material.

For example, woolen fibers can be treated by blending the wool with cellulosics thus making the resultant fibers hydrophobic. In addition or alternatively, durable metallic salts such as Orco Repel S from ORCO (Organic Dyes and Pigments) LLC of Lincoln, R.I. can be blended with the wool to create hydrophobic fibers.

In some examples, the second side of the mesh interface comprises the hydrophilic fiber(s) in a predetermined amount configured to provide a comfortable feel on the patient's skin. For example, the second side of the mesh interface comprises about 15 weight percent to about 85 weight percent, or about 25 weight percent to about 60 weight percent, or about 30 weight percent to about 50 weight percent of the hydrophilic fiber(s) on a basis of total weight of the hydrophilic fiber(s), hydrophobic fiber(s) and the conductive fiber(s), and the conductive particles (if present).

The hydrophilic fibers incorporated into the second side of the mesh interface 70, 570 can be configured to have a soft, comfortable feel and are able preferably to absorb moisture on the patient's skin and wick the moisture away from the patient's skin, which further reduces the potential for abrasion and irritation of the patient's skin. Additionally, the nylon, polyester, and/or cotton fibers are more breathable than the conductive fibers or particles 74, 574 and trap less heat against the patient's skin, which increases overall comfort to the patient P. The nylon, polyester, and/or cotton fibers can also protect the conductive fibers or particles 74, 574 from wear and external damage, which can prolong the operational life of the support garment 20. According to an example, the hydrophilic fibers 73 comprise a textured nylon yarn, which creates a soft surface at the first side 71 and the second side 72 of the mesh interface 70.

In some examples, at least one of the hydrophobic fiber(s) and/or the hydrophilic fiber(s) can comprise microfibers. The decreased filament size provides greater surface area, thereby having more pores to transport vapor by capillary action. Higher pore density can provide better thermoregulation. Knitwear or woven fabric produced using microfibers provides soft, lightweight and comfortable fabrics. Also, microfibers exhibit better moisture control and transport. Commonly produced microfibers include polyester, nylon, rayon, and acrylic, and can be blended with natural fibers such as silk, cotton, and wool. In textile structures, microfibers packed tightly together form narrow capillaries, enabling fabrics to transport liquids more readily than those made from conventional sized fibers. Examples of polyester or nylon microfibers include those available from Tricol (Weifang Tricol Textile Co. Ltd.) and such as are used in the Dri-Fit technology used by Nike. In some examples, the weight percentage of microfibers can comprise about 1 to about 75 weight percent, or about 5 to about 50 weight percent, or about 10 to about 25 weight percent, on a basis of total weight of the hydrophobic fiber(s) or the hydrophilic fiber(s), respectively.

In some examples, the mesh interface can comprise one or more hydrophobic and/or hydrophilic fibers which are dielectric. As described herein, dielectric materials or fibers can be electrically non-conductive, poor conductors of electricity, transmit electric force without conduction, or act as an electrical insulator. In some examples, dielectric materials or fibers can be poor conductors of electricity, yet have the capacity to store energy by polarization or are capable of supporting an electric field, which may improve transmission of the defibrillation therapy. In some examples, the electrical resistivity of the dielectric fiber can be about 10¹¹ ohm meters (Ω m) or more, or about 10¹¹ Ω m to about 10¹⁹ Ω m at a temperature of about 25° C. In some examples, dielectric materials or fibers can comprise at least one nonmetallic material. In some examples, the dielectric fibers can be formed from materials comprising one or more of polyester, polypropylene, olefin, acrylic, modacrylic, silk, nylon, spandex, bamboo, cotton, wool, linen, acetate, cellulosic, rayon, and combinations thereof.

In some examples, the mesh interface can comprise one, more, or all hydrophobic and/or hydrophilic fibers (excluding the conductive fibers discussed herein) which are not dielectric, for example the hydrophobic and/or hydrophilic fiber(s) can have an electrical resistivity of less than about 10¹¹ Ω m, or about 10⁻⁶ to about 10⁶ Ω m (semiconductors) at a temperature of about 25° C.

In some examples, the mesh interface comprises conductive fiber(s) and/or conductive particles configured to be interspersed with the hydrophobic fiber(s) and with the hydrophilic fiber(s), such that the conductive fiber(s) and/or conductive particles conduct therapeutic electrical current from the therapy electrode to the patient's skin, e.g., the conductive fiber(s) and/or conductive particles are configured to form a plurality of conductive pathways extending from the first side of the mesh interface to the second side of the mesh interface.

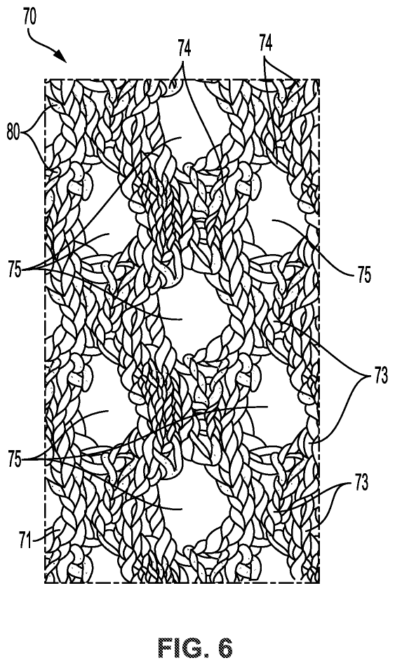

In some examples, the conductive fibers and/or conductive particles 74 comprise a conductive yarn 77, such as a silver-plated nylon yarn, which is interlaced, such as by knitting or weaving, with the hydrophobic and/or hydrophilic fibers, as will be discussed in further detail below with reference to FIGS. 6-8. According to another example, the conductive fibers and/or conductive particles 74 can be comprised of a liquid or gel based coating, i.e., paint, powder coating, and/or other fine material coating of conductive material, e.g., metallic particles such as silver particles, applied to a layer or layers of dielectric material in a concentrated manner at select locations to saturate the dielectric material through its thickness. In some examples, polymer fiber(s) such as the hydrophobic fiber(s) and hydrophilic fiber(s) discussed herein can be made conductive by loading a conductive filler before forming or coating with conductive material after forming the fiber or yarn.

According to another example, the conductive fibers and/or conductive particles 74 comprise concentrated tufts or wads of a conductive yarn or other conductive fabric material inserted or plugged into a dielectric fabric material so as to extend through the thickness of the dielectric fabric material. According to another example, the conductive fibers and/or conductive particles 74 comprise conductive wires inserted into, such as by embroidering, a textile or fabric substrate.

According to an example of the present disclosure, the mesh interface 70, 570 is configured to provide an electrical impedance of the plurality of conductive pathways 80, 580 extending through the mesh interface 70, 570 from the first side 71, 571 to the second side 72, 572 of about 0.01Ω to about 20Ω, or about 0.01Ω to about 10Ω, or about 0.01Ω to about 5Ω, or about 0.1Ω to about 2Ω, or about 0.25Ω to about 1.5Ω. It is to be appreciated that the mesh interface 70, 570 can provide any suitable electric impedance of the plurality of conductive pathways 80.

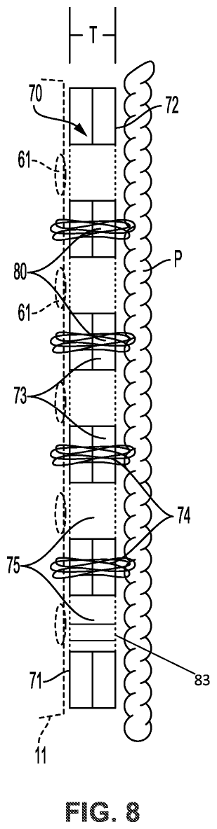

The electrical impedance values noted herein can be determined based on a mannequin test carried out in a following manner. For example, as shown in FIGS. 9A and 9B, a garment 1600 implementing the mesh interface 1603 can be positioned over a mannequin 1602. The mesh interface material 1603 shall be new and unwashed. To perform the test, the garment 1600 is placed on the mannequin 1602 as shown in FIGS. 9A and 9B. Therapy electrodes 1604 are placed in front 57 and rear pockets 56a that each include mesh interfaces 1603. A conductive pad 1606 is then placed under the garment 1600, between the garment 1600 and the surface of the mannequin 1602, such that the conductive pad 1606 is disposed between the mesh interfaces 1603 and the surface of the mannequin 1602. The conductive pad 1606 is connected by an electrical connector 1609 to wires to complete a circuit 1607 with one of the therapy electrodes 1604, which is also connected by an electrical connector 1609 to the wires forming the circuit 1607. Initially, a test current can be applied to the circuit. For example, the test current can be of predetermined suitable current parameters determined by circuit design. For example, the test current can comprise 0.01 A within a range of 200 Hz-200 MHz. Prior to an impedance measurement, conductive gel in a predetermined suitable quantity is deployed between the therapy electrode and the conductive pad as noted below. An ohmmeter and/or an A.C. bridge 1608 can be connected within the circuit 1607 and configured to measure the resistance of the mesh interface 70. In one scenario, the test can be performed at room temperature which corresponds to normal operating circumstances of the device. In some scenarios, the test can be repeated at a range of temperatures corresponding to usual ambient and/or typical patient use environments. For example, the test can be performed at temperatures ranging from −15 F to around 120 F. For example, the test can be performed at between 20% and 95% relative humidity. In some scenarios, typical ranges for carrying out the test can be within the range of 0° C. to 50° C. (32° F. to 122° F.), up to 95% relative humidity (non-condensing), and between sea level and up to 10,000 feet in altitude. The above-mentioned mannequin test is implemented to test the impedance each of mesh interface 1603 individually. It is to be appreciated that similar tests may be performed to test the impedance values of two or more mesh interfaces 1603 collectively.

According to an example, the plurality of conductive fibers and/or conductive particles 74, 574 comprises an impedance measure of about 10 Ω/meter to about 250 Ω/meter, or about 20 Ω/meter to about 150 Ω/meter, or about 30 Ω/meter to about 130 Ω/meter. It is to be appreciated that the plurality of conductive fibers or particles 74 can comprise an impedance measure of any suitable value.

The mesh interface 70, 570 is configured to provide a comfortable feel on the patient's skin and to wick moisture away from the patient's skin. As discussed above, the implementations herein include conductive metallic materials incorporated into the mesh interface 70, 570 to reduce abrasion and irritation of the patient's skin, particularly during continuous use of the support garment 20 during an extended period of time. Also, such implementations may cause fewer negative reactions, such as an allergic reactions, to the metallic conductive materials. According to the example, the mesh interface 70, 570 incorporates a reduced amount of the conductive metallic material, thereby reducing the potential for abrasion and irritation of the patient's skin, as well as the potential for negative reactions in response to prolonged contact with the mesh interface 70, 570. In some examples, the metallic material comprises silver metal. For example, silver metal comprises better conductivity than most other conductive metals, and further is more tolerable on skin than other conductive metals. In some examples, silver comprises natural antimicrobial properties. Examples of such conductive materials for use in mesh interface as described in further detail below.

In some examples, the mesh interface can comprise fusible fiber(s) 76, 576 (shown for example in FIGS. 14 and 16) in addition to the above-mentioned hydrophobic fibers(s), hydrophilic fiber(s) and/or conductive fiber(s) disclosed herein. The fusible fibers are configured to melt, dissipate, and/or shrink in volume relative to the conductive fibers or particles 74, 574 and the other fibers 73a, 73b, 573a, 573b when exposed to heat, such as heat from steam generated by a garment steamer at about 70° C.-160° C. According to an example, the fusible fibers comprise a low melt, thermoplastic material, such as low melt nylon and/or low melt polyester materials. According to an example, the fusible fibers comprise a fusible bonding yarn formed from low melt nylon and/or low melt polyester multifilaments.

The fusible fibers provide for a good hand feel to the mesh interface 70, 570. Also, as will be discussed in additional detail below, heating of the fusible fibers, thus causing the fusible fibers to melt, dissipate, and/or shrink in volume, results in the conductive fibers or particles 74, 574 expressing more relative to the hydrophobic fibers(s) and/or hydrophilic fiber(s), whereby the plurality of conductive pathways 80, 580 extending through the mesh interface 70, 570 project from the first side 71, 571 and the second side 72, 572 of the mesh interface 70, 570, as shown in FIGS. 7 and 8. According to the example, the shrinkage of the fibers 73a, 73b, 573a, 573b and the projection of the conductive pathways 80, 580 from the first side 71, 571 and the second side 72, 572 of the mesh interface 70, 570 allows for the number of conductive fibers or particles 74, 574 to be reduced within the mesh interface 70, 570 while achieving the sufficiently low impedance to effectively transmit the therapeutic electrical pulses from the therapy electrodes 11 to the patient P. The reduction in the amount of conductive fibers or particles 74, 574 in the mesh interface allows for increased comfort and the potential for less skin irritation experienced by the patient P.

The mesh interface 70, 570 is comfortable so as not to cause human skin irritation after predetermined test periods as set forth below (e.g., after 1 day of continuous contact exposure, after 2 days of continuous contact exposure, or after 3 days of continuous contact exposure). For instance, in some examples, the mesh interface 70, 570 is constructed so as to score zero or no more than one on the Human Skin Irritation Test set forth in Annex C of the ANSI/AAMI/ISO 10993-10:2010 standards for Biological Evaluation of Medical Devices—Part 10: Tests for Irritation and Skin Sensitization. Table C.1 of Annex C, which provides the grading scale for the Human Skin Irritation Test, is set forth below. In accordance with ISO 10993-10 C3.3., at least 30 volunteers shall complete the test, with no less than one-third of either sex. The mesh interface test material shall be applied to intact skin at a suitable site, e.g. the upper outer arm. The application site shall be the same in all volunteers and shall be recorded. Generally, the mesh interface test material shall measure at least 1.8 cm, preferably 2.5 cm in diameter. The mesh interface test material shall be held in contact with the skin by means of a suitable non-irritating dressing, including non-irritating tape, for the duration of the exposure period. In one scenario, the mesh interface test material can be pre-moistened with water before application. To avoid unacceptably strong reactions, a cautious approach to testing shall be adopted. A sequential procedure permits the development of a positive, but not severe, irritant response. The mesh interface test materials are applied progressively starting with durations of 15 min and 30 min, and up to 1 h, 2 h, 3 h and 4 h. The 15 min and/or 30 min exposure periods may be omitted if there are sufficient indications that excessive reactions will not occur following the 1 h exposure. If no reaction or no excessive reactions are observed, the duration can be increased to 1 day, 2 days, and 3 days. Progression to longer exposures, including 24 h exposure at a new skin site, will depend upon the absence of skin irritation (evaluated up to at least 48 h) arising from the shorter exposures, in order to ensure that any delayed irritant reaction is adequately assessed.

Application of the material for a longer exposure period is always made to a previously untreated site. At the end of the exposure period, residual test material shall be removed, where practicable, using water or an appropriate solvent, without altering the existing response or the integrity of the epidermis. Treatment sites are examined for signs of irritation and the responses are scored immediately after mesh interface test material removal and at (1±0.1) h to (2±1) h, (24±2) h, (48±2) h and (72±2) h after patch removal. If necessary to determine reversibility of the response, the observation period may be extended beyond 72 h. In addition, the condition of the skin before and after the test shall be thoroughly described (e.g. pigmentation and extent of hydration). Skin irritation is graded and recorded according to the grading given in Table C.1 of Annex C of ANSI/AAMI/ISO 10993-10:2010 standards for Biological Evaluation of Medical Devices—Part 10: Tests for Irritation and Skin Sensitization.

TABLE C.1
Human skin irritation test, grading scale

| Description of response | Grading |
|---|---|
| No reaction | 0 |
| Weakly positive reaction (usually characterized by mild | 1 |
| erythema and/or dryness across most of the treatment site) | |
| Moderately positive reaction (usually distinct erythema or | 2 |
| dryness, possibly spreading beyond the treatment site) | |
| Strongly positive reaction (strong and often spreading | 3 |
| erythema with oedema and/or eschar formation) | |

The mesh interface 70, 570 can also be configured to facilitate a transfer of electrically conductive fluid 83 from the at least one therapy electrode 11 to the patient's skin. As shown in FIGS. 5-8 and 13, the mesh interface 70, 570 can further comprise one or more opening(s) 75, 575 extending through the mesh interface 70, 570 from the first side 71, 571 to the second side 72, 572. The mesh interface 70, 570 is configured to facilitate transfer of electrically conductive fluid 83 from the at least one therapy electrode 11 to the patient's skin via the opening(s) 75, 575.

In some examples, the mesh interface 70, 570 is configured to receive electrically conductive fluid 83 from the electrically conductive fluid deployment opening(s) 117 in the at least one therapy electrode 11 in an amount of about 0.1 cubic-centimeter (cc) to about 100 cc, or about 0.1 cubic-centimeter (cc) to about 75 cc, or about 0.1 cubic-centimeter (cc) to about 30 cc of electrically conductive fluid. In examples, the mesh interface 70, 570 is configured to receive electrically conductive fluid 83 from the electrically conductive fluid deployment opening(s) 117 in an amount of 0.1 cubic-centimeter (cc) to about 30 cc, about 0.5 cc to about 20 cc, about 0.9 cc to about 10 cc, or 0.9 cc to 5 cc. It is to be appreciated that the mesh interface 70, 570 can be configured to receive any suitable amount of the electrically conductive fluid. FIG. 8 depicts the electrically conductive fluid deployment opening(s) 117 of the therapy electrode 11 in alignment with the opening(s) 75 of the mesh interface. This depiction is provided as an example and done for ease of illustration and to demonstrate the purpose of the opening(s) 75, 575 to facilitate transfer of electrically conductive fluid 83 from the electrically conductive fluid deployment opening(s) 117 in the therapy electrode 11 to the patient P. It is not necessary for the mesh interface 70, 570 to be configured in such a manner such that the opening(s) 75, 575 in the mesh interface align with the electrically conductive fluid deployment opening(s) 117 in the therapy electrode 14. In some examples, the diameter of each opening 75, 575 can be independently selected from up to about 0.5 inch, or about % inch to about 0.5 inch, or about 3/16 inch to about 0.5 inch, about ⅜ inch to about 0.5 inch, 0.5 inch, % inch, 3/16 inch or ⅜ inch. In some examples, each of the opening(s) independently has an average diameter of about 0.005 inches to about 0.3 inches (about 0.13 mm to about 7.6 mm).

In some examples, at least a portion of the opening(s) 75, 575 are aligned with respective electrically conductive fluid deployment opening(s) 117 on the therapy electrode to facilitate transfer of the electrically conductive fluid 83 from the at least one therapy electrode 14 to the patient's skin via the opening(s) 75, 575. In some examples, at least a portion of the opening(s) are formed by spaces between hydrophobic dielectric fiber(s)/yarn(s), hydrophilic dielectric fiber(s)/yarn(s), and/or conductive fiber(s)/yarn(s), and fusible fiber(s)/yarn(s) (if present).

In some examples, the mesh interface 70, 570 is further configured to be porous to the electrically conductive fluid 83 by a plurality of secondary openings within the mesh interface 70, 570, e.g., the interstices between interwoven yarns and fibers.

According to an example, the mesh interface 70, 570 is configured to be porous to the electrically conductive fluid from openings 117 in the at least one therapy electrode 11 to provide a predetermined electrical impendence of the plurality of conductive pathways 80 extending through the mesh interface 70, 570 from the first side 71 to the second side 72 of about 0.01Ω to about 0, or about 0.010 to about 10Ω, or about 0.01Ω to about 5Ω, or 0.01Ω to 5Ω, or about 0.1Ω to about 2Ω, or about 0.25Ω to about 1.5Ω. It is to be appreciated that the mesh interface 70, 570 can be porous to the electrically conductive fluid 83 to provide a predetermined electrical impedance of any suitable value. The electrical impedance values noted herein can be determined based on a test carried out in a manner that is similar to that described above as the mannequin test.

The plurality of openings 75, 575 are provided in the mesh interface 70, 570 in a sufficient number and have a sufficient size to facilitate the transfer of a suitable amount of the electrically conductive fluid 83 from the at least one therapy electrode 11 to the patient's skin to achieve an appropriate level of impedance (or alternatively measured as admittance, which is the inverse of impedance) between the at least one therapy electrode 11 (via the mesh interface 70, 570) and the patient's skin such that the therapeutic electrical pulses are delivered to the patient's heart without burning or with minimal burning of the patient's skin. For example, the appropriate level of impedance between the at least one therapy electrode 11 via the mesh interface 70, 570 and the patient's skin is tested per the mannequin test as noted above, and is about 0.01Ω to about 20Ω, including ranges therebetween as noted above.

According to an example, the plurality of openings 75, 575 comprises about 2 to about 1000 openings per square inch of the mesh interface 70, 570, or about 5 to about 500 openings per square inch, or about 10 to about 100 openings per square inch. It is to be appreciated that the plurality of openings 75, 575 can comprise any suitable number of openings. According to an example, the plurality of openings 75, 575 have an average diameter in a range of about 0.005″ to about 0.3″ (about 0.13 mm to about 7.6 mm), or about 0.01″ to about 0.2″ (about 0.25 mm to about 5.1 mm), or about 0.05″ to about 0.1″ (about 1.3 mm to about 2.5 mm). It is to be appreciated that the plurality of openings 75, 575 can have any suitable average diameter or range of varying average diameters.

According to an example, the plurality of openings 75, 575 can have a non-circular shape, such as quadrilateral, rectangular, square, triangular, pentagonal, hexagonal, octagonal, etc. According to the example, the plurality of openings can have an average area in a range of about 0.01 mm² to about 45.4 mm², or about 0.05 mm² to about 20.4 mm², or about 1.3 mm² to about 4.9 mm².

The thickness T, shown in FIG. 8, of the mesh interface 70, 570 can also affect the amount of the electrically conductive fluid transferred from the at least one therapy electrode to the patient's skin. The thickness T is measured prior to application to the patient's body as the mesh interface material will be compressed between the therapy electrode and patient's skin. According to an example, a thickness T of the mesh interface 70—(in uncompressed form) from the first side 71 to the second side 72, shown in FIG. 8, is about 0.005″ to about 0.5″ (about 0.13 mm to about 12.7 mm), or about 0.01″ to about 0.25″ (about 0.25 mm to about 6.4 mm), or about 0.03″ to about 0.1″ (about 0.76 mm to about 2.5 mm). It is to be appreciated that the mesh interface 70, 570 can be provided with any suitable thickness T in order to carry out its functionality as described herein.

With reference to FIGS. 6-8, 10, 13, 14, and 16, the mesh interface 70, 570, can be formed by intertwining together the hydrophobic fiber(s), hydrophilic fiber(s), conductive fiber(s) or particles, and fusible fiber(s) (if present), any of which can be in the form of yarn. It is to be appreciated that the fiber(s)/yarn(s) can be intertwined in any suitable manner. For example, the mesh interface 70, 570 can be formed by a knitting, weaving, and/or interlacing process, or any other suitable process for creating a fabric material. The process of intertwining the fiber(s)/yarn(s) can be performed automatically by a suitable machine.

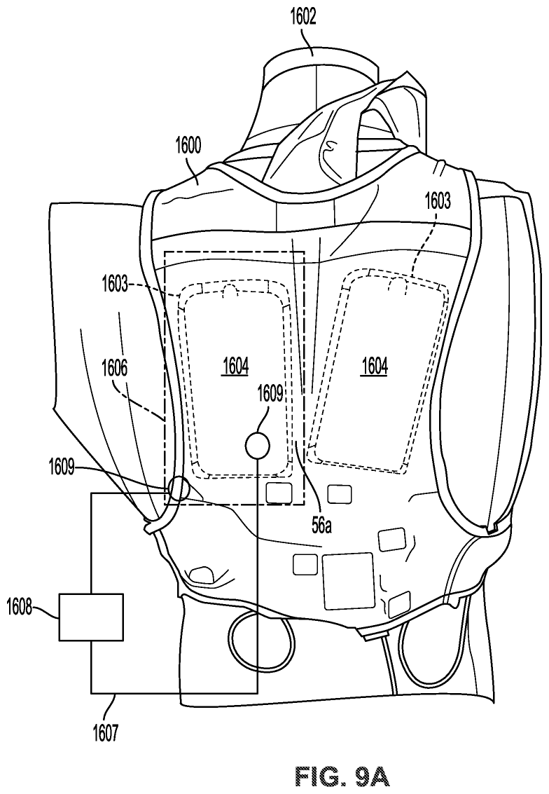

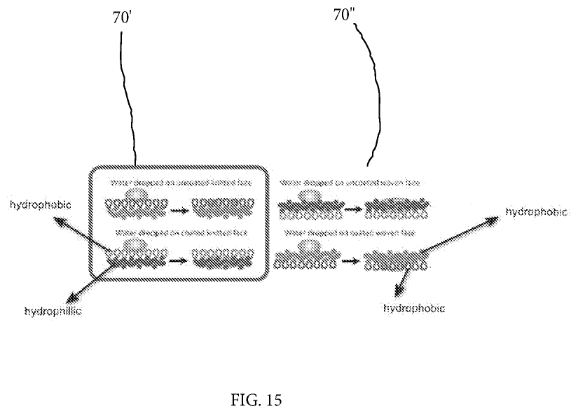

In some examples, the fiber(s)/yarn(s) yarn can be knit together by flat or circular knit plating. A plated knit structure is shown generally in FIGS. 10 and 13. The plating process forms a first layer on a first side of the knit (technical face), and another layer on a second side of the knit (technical back). One or more additional layers can be provided between the first and second layers. The knitting hook of FIG. 17 shows the relative position of the yarn with respect to the layers formed. For example, the technical face or first side of the knit can be formed from the hydrophobic fiber(s)/yarn(s) and the technical back or second side of the knit can be formed from the hydrophilic fiber(s)/yam(s). In some examples, such as shown in FIG. 17, one or more conductive fiber(s)/yam(s) can be positioned on the hook between the hydrophobic fiber(s)/yarn(s) and the hydrophilic fiber(s)/yarn(s). The number and order of the types of fiber(s)/yarn(s) can be varied between the outer hydrophobic fiber(s)/yam(s) that form the first side and the outer hydrophilic fiber(s)/yarn(s) that form the second side.

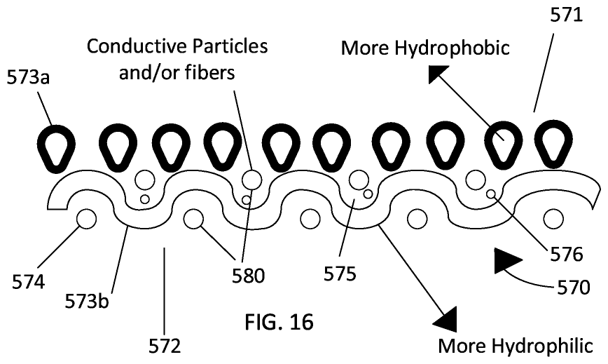

A plated structure contains loops composed of two, or three (or more) yarns. Each yarn has been separately supplied through its own guide or guide hole to the needle hook, in order to influence its respective position relative to the surface (technical face and technical back of the fabric). The basic rule of plating is that the yarn positioned nearest to the needle head shows on the reverse side of the needle loop and therefore shows on the surface of the technical back (FIG. 18). The second yarn is in a lower position and tends to show on the face stitches of weft- and warp-knitted structures. The second yarn will be prominent on the surface of face loops on both sides of rib fabrics unless it is tucked (‘tuck plated’) by the second set of needles. In purl fabrics, face stitches will show the second yarn and reverse stitches the first yarn.

In some examples, the fiber(s)/yarn(s) yarn can be knit together to form a spacer fabric, which is a double-faced fabric knitted on a double bar machine. The distance between the two surfaces is retained after compression by the resilience of the pile yarn that passes between them. Weft-knitted space fabrics are manufactured using a circular or a flat knitting machine. Two surface layers are manufactured on two needle beds respectively, and they are connected by tuck stitch. The distance between two needle beds can be adjusted to produce different thicknesses according to the requirement. At present, weft-knitted space fabrics are usually manufactured using an automatic flat knitting machine. Double faced fabrics can be produced on dial and cylinder, v-bed and purl machines.

Two surface layers are manufactured using one or two guide bars on two needle beds. The texture depends on their structure. If the structure is compact, two bar tricot stitch or lock-knit stitch can be used. If the surface is net-structured, some guide needles can be empty. If the surface possesses a figured effect, one guide bar produces ground texture, while the other one produces the pattern. The space layer connects one surface with the other, and keeps a distance between them. Therefore, space yarn must possess good stiffness, and its fineness depends on the space distance. The space yarn forms the loops at the front and the back beds in turn to connect the two surface layers together. The distance between the two surface layers depends on the distance between the front and the back beds.

According to one example, the dielectric yarn or yarns and the conductive yarn can be flat knitted on E7.2 Stoll ADF32-W knitting machine having front and back needle beds in a V-Bed configuration, which is commercially available from STOLL of Reutlingen Germany. It is to be appreciated that the mesh interface 70 can be machine knitted on a suitable machine according to any suitable technique, particularly on a suitable 14 gauge double bed flat knitting machine.

In examples, the knitting machine is configured to be controlled by a processor executing a plurality of machine-readable instructions stored on a non-transitory computer-readable medium. In implementations, knitting yarn into a seamless knitted preform using a computerized flat knitting machine allows for variations in shapes that can be produced with a reduced amount of materials and parts waste, human effort, and time. For example, a user can design the therapy mesh interface in accordance with the principles described herein based on a three-dimensional shape using a computer-aided design (CAD) program. The design can then be knit into a seamless knitted preform by the computerized flat knitting machine such that multiple sheets of materials and yarn do not need to be manually cut and laid up to form the shape and structure of the therapy mesh interface. In examples, the processor can be disposed within a printed circuit board (PCB), and can comprise an integrated random operating memory (ROM) chip. As controlled by a custom run program stored on the processor to implement the therapy mesh interface described herein, the processor generates control signals to engage the drive motion in coordination with the mechanical spring arm of the knitting machine.

According to one example, the textured hydrophobic yarn comprises 70/1/34 denier textured Unifi Resist2O yarn (https://unifi.com/innovations/resist2o), the textured hydrophilic yarn comprises 70/1/34 denier textured Unifi Sorbtek yarn (https://unifi.com/innovations/sorbtek) and the fusible yarn comprises 75 deci-tex/F75 fusible nylon yarn. According to an example, the fusible yarn comprises a low melt, thermoplastic material, such as multifilaments of low melt nylon and/or low melt polyester, and is formed such that the fusible yarn begins to melt, dissipate, and/or shrink in volume when exposed to heat at 75° C.

According to an example, the conductive yarn 77 comprises a silver-plated nylon yarn. The silver-plated nylon yarn can be a 2 ply 100 denier conductive yarn with a maximum resistance of 75 Ω/meter. According to the example, the silver material is chosen for the conductive yarn 77 due to its conductivity and biocompatibility, i.e., reduced potential for skin irritation and negative reactions. According to the example, the silver-plated nylon yarn can be generated according to any plating or metal application technique for depositing the silver material on the nylon (or other polyamide) substrate material, including through the use of electrolysis, chemical reactants, bundle drawing, machining, lamination, foil-shaving, and/or metalizing/vapor deposition.

According to an example, a conductive yarn 77 different from the silver-plated nylon yarn can be intertwined with the hydrophobic yarn and the hydrophilic yarn. For instance, the conductive yarn 77 can comprise a nickel plated/metalized or aluminum plated/metalized nylon, other polyamide, or polytetrafluoroethylene (PTFE) yarn. The conductive yarn 77 can also comprise a carbon coated or carbon filled yarn or filament material. Alternatively, the conductive yarn 77 can comprise a yarn material that has been coated or painted with a conductive paint material, such as a silver loaded polymer paint.

According to an example, the mesh interface 70, 570 comprises about 10% to about 60% by weight of conductive yarn 77, or about 15% to about 54% by weight of conductive yarn 77, or about 20% to about 35% on a basis of total weight of the hydrophobic fiber(s)/yarn(s), hydrophilic fiber(s)/yarn(s), and conductive yarn(s). It is to be appreciated that the conductive yarn 77 can be provided in any suitable amount or ratio. In particular, the mesh interface 70, 570 incorporates a sufficient amount of conductive yarn 77, 577 to form sufficient conductive pathways 80, 580 extending through the mesh interface 70, 570 from the first side 71, 571 to the second side 72, 572 such that the mesh interface 70, 570 has a sufficiently low impedance to allow for the therapeutic electrical pulses to be conducted from the at least one therapeutic electrode 11 to the patient's skin P.

The impedance of the mesh interface 70, 570 must be maintained over the operational life of the support garment 20 through continuous or nearly continuous use and multiple wash cycles. For reference, patients can be instructed to wash their support garments every day or nearly every day. Accordingly, 30 wash cycles represents an approximate typical number of wash cycles of the support garment for a month. Continuous use of the support garment and multiple wash cycles tend to result in the loss of conductive metal material from the mesh interface 70, 570 over time, resulting in an increase in the impedance of the mesh interface 70, 570 over time.

Examples of the mesh interface 70, 570 having a higher weight percentage of the conductive yarn 77 can be associated with support garments 50 that are intended to have a longer operational life since the presence of a sufficient amount of conductive metal material can be maintained in the mesh interface 70, 570 through continuous wear of the support garment 20 and a large number of wash cycles, i.e., 30 or more days/wash cycles.

Examples of the mesh interface 70, 570 having a lower weight percentage of the conductive yarn 77 can be associated with support garments 50 that are intended to have a more limited operational life, i.e., 10-15 days/wash cycles, as the impedance of the mesh interface 70, 570 will not be maintained over time due to the loss of the conductive material from the mesh interface after a short amount of time. According to an example, a support garment 20, which is intended to be worn for a limited amount of time, i.e., no more than 10-15 days, can be provided with a mesh interface 70, 570 including a reduced weight percentage of the conductive yarn 77 to reduce cost and avoid waste.

As shown in FIGS. 10 and 13, the mesh interface 70 can comprise a plurality of intertwined structures of the hydrophobic fiber(s)/yam(s), hydrophilic fiber(s)/yarn(s), and conductive yarn(s). According to an example, the plurality of intertwined structures comprises a pattern of at least three intertwined structures. The at least three intertwined structures comprise a plurality of courses of the hydrophobic fiber(s)/yam(s) arranged in a tubular pattern structure; at least one course of the conductive yarn 77 intertwined with the plurality of courses of the hydrophobic fiber(s)/yam(s) in a lxi rib pattern structure; and at least one pointelle pattern structure of intertwined hydrophilic fiber(s)/yarn(s). In examples, pointelle includes a knit fabric structure in which the knitting process forms a pattern of small holes in the finished fabric material.

As discussed above, the hydrophobic fiber(s)/yarn(s), hydrophilic fiber(s)/yarn(s), and the fusible yarn can be arranged together in a plated yarn structure. In other words, the hydrophobic fiber(s)/yarn(s), hydrophilic fiber(s)/yarn(s), and the fusible yarn can be fed to the needle bed together or in a slightly offset manner such that both yarns are knitted together course by course with one yarn, i.e., the hydrophobic fiber(s)/yarn(s), showing on the needle front (technical face) and one yarn, i.e., the hydrophilic fiber(s)/yarn(s), showing on the needle back (technical back).

With reference to FIG. 5, according to an example, the mesh interface 70 can include a central portion 78 including hydrophobic fiber(s)/yam(s) and hydrophilic fiber(s)/yarn(s) and the conductive yarn 77 intertwined with each other in the tubular pattern structures, the 1×1 rib pattern structures, and the pointelle pattern structures described above. A peripheral portion 79 can be formed surrounding the central portion 78 that does not include the openings 75 formed by the pointelle pattern structures. The peripheral portion 79 can include the hydrophobic fiber(s)/yarn(s) and hydrophilic fiber(s)/yarn(s) and the conductive yarn 77 intertwined with each other in the tubular pattern structures A and the 1×1 rib pattern structures B described above. The peripheral portion 79 of the mesh interface 70, 570 can be less porous with respect to the passage of the conductive gel from the plurality of openings 117 in the therapy electrode 11 to the patient's skin P than the central portion 80 of the mesh interface 70, while remaining configured to conduct the therapeutic electrical pulses from the therapy electrode 11 to the patient P.

According to another example, the peripheral portion 79 may not include any conductive yarn 77 or conductive fibers and/or conductive particles 74 and may not contribute to conduction of the therapeutic electrical pulses from the therapy electrode 11 to the patient P. Rather, the peripheral portion 79 may be formed entirely from dielectric materials. According to the example, the peripheral portion 79 can be formed from the hydrophobic fiber(s)/yam(s) and hydrophilic fiber(s)/yarn(s) and fusible yarn intertwined as described above herein and incorporating a pointelle pattern to form openings in the peripheral portion 79 that allow for conductive gel to pass through the peripheral portion 79 from the therapy electrode 11 to the patient's skin P. Alternatively, the peripheral portion 79 can be formed from an entirely different knitted pattern structure surrounding the central portion 78 or from an entirely different fabric material that the central portion 78 can incorporated into, such as by stitching, as a patch. According to another example, the entire mesh interface 70 is formed entirely of the tubular pattern structures, the 1×1 rib pattern structures, and the pointelle pattern structures.

In implementations, each mesh interface 70 can be constructed in a different manner or each mesh interface 70 can be identical. For example, referring to FIG. 4A, one or both rear mesh interfaces 70a and 70b can be implemented with more metallic material than the front mesh interface 70c. For example, referring to FIG. 4A, one or both rear mesh interfaces 70a and 70b can be implemented metallic material and no dielectric material, while the front mesh interface 70c can be implemented with a mix of metallic material and dielectric material as described herein. According to an example, the mesh interfaces 70a, 70b of the support pockets 56a, 56b in the back portion 51 of the support garment 20 are made entirely from conductive yarn and the mesh interface 70c of the support pocket 57 in the belt portion 22 of the support garment is formed according to the structure described herein. Alternatively, the mesh interfaces 70a, 70b in the back portion 51 can include a higher content of conductive yarn than the mesh interface 70a in the belt portion 22 to achieve a lower impedance. Such implementations can be more feasible without increasing the skin irritation of the patient P, because the back portion 51 of the support garment 20 is less likely to move with respect to the patient's skin than the belt portion 22, which tends to be shifted in position as the patient lies down, sits, stands, raises his/her arms, moves, bends, etc., thus causing additional friction between the mesh interface 70 and the patient's skin.

As discussed above, the dielectric fibers 73 of the mesh interface 70 include hydrophobic fiber(s)/yarn(s) and hydrophilic fiber(s)/yarn(s) and a fusible yarn 76 that is configured to melt, dissipate, and/or shrink in volume relative to the nylon or cotton yarn and the conductive yarn when exposed to heat, such as steam generated by a garment steamer at 70° C.-160° C. Accordingly, heating of the hydrophobic fiber(s)/yarn(s) and hydrophilic fiber(s)/yarn(s) of the mesh interface causes the fusible yarn to melt, dissipate, and or shrink in volume thereby contracting the structures A of hydrophobic fiber(s)/yarn(s) and hydrophilic fiber(s)/yarn(s) relative to the structures B of conductive yarn 77 in the mesh interface 70, which enhances the standing out of the conductive yarn 77 from the first and second sides 71, 72 of the mesh interface 70.

FIG. 10 illustrates an example mesh interface 70 prior to being exposed to an application of heat during design and manufacture. The hydrophobic fiber(s)/yarn(s) and hydrophilic fiber(s)/yarn(s) and the fusible yarns are relatively relaxed prior to the application of heat. In the heating process, the fusible yarns melt, dissipate, and/or shrink in volume, thus contracting the hydrophobic fiber(s)/yarn(s) and hydrophilic fiber(s)/yarn(s) structures, which has the effect of widening the plurality of openings 75. This effect allows for greater electrical connectivity between the conductive fibers or particles 74 interspersed within the mesh interface 70, thereby resulting in an increased plurality of conductive pathways through the mesh interface 70 from the first side 71 to the second side 72. For example, the heat process can cause the conductive fibers or particles to express more relative to the hydrophobic fiber(s)/yarn(s) and hydrophilic fiber(s)/yarn(s). The hydrophobic fiber(s)/yarn(s) and hydrophilic fiber(s)/yarn(s) recede during the process to be sub-flush relative to the conductive fibers. This process allows for fewer conductive fibers or particles to be used in the development of the mesh interface. Accordingly, stretching and application of heat to the mesh interface 70 can be utilized to reduce conductive fibers or particles, and improve transfer of conductive fluid and conduction of therapeutic electrical pulses through the mesh interface 70 from the at least one therapy electrode 11 to the patient P.

The features of the wearable cardiac therapeutic device, support garment, and mesh interface of the present disclosure can encourage patients to wear the support garment and associated medical device for longer and/or continuous periods of time with minimal interruptions in the periods of wear. For example, by minimizing interruptions in periods of wear and/or promoting longer wear durations, patients and caregivers can be assured that the device is providing desirable information about as well as protection from adverse cardiac events such as ventricular tachycardia and/or ventricular fibrillation, among others. Moreover, when the patient's wear time and/or compliance is improved, the device can collect information on arrhythmias that are not immediately life-threatening, but can be useful to monitor for the patient's cardiac health. Such arrhythmic conditions can include onset and/or offset of bradycardia, tachycardia, atrial fibrillation, pauses, ectopic beats bigeminy, trigeminy events among others. For instance, episodes of bradycardia, tachycardia, or atrial fibrillation can last several minutes and/or hours. The wearable cardiac therapeutic device, support garment, and mesh interface disclosed herein provide features that encourage patients to keep the device on for longer and/or uninterrupted periods of time, thereby increasing the quality of data collected about such arrhythmias. Additionally, features as described herein promote better patient compliance resulting in lower false positives and noise in the physiological signals collected from ECG electrodes and other sensors disposed within the support garment. For example, when patients wear the device for longer and/or uninterrupted periods of time, the device tracks cardiac events and distinguishes such events from noise over time.

The improvements incorporated in the support garment can provide comfort and wearability to the patient by utilizing softer materials for at least some of the components of the support garment and by utilizing materials and construction features that are less likely to dig into and/or rub on the patient's skin in a painful or irritating manner. These features can encourage patients to wear the support garment for longer and/or continuous periods of time to assure that the device is providing desirable information about as well as protection from adverse cardiac events such as ventricular tachycardia and/or ventricular fibrillation, among others.

Aspects of the present disclosure are directed to monitoring and/or therapeutic medical devices configured to identify a patient physiological event and, in response to the identified event, to provide a notification to the patient wearing the device. The notification can include an instruction or request to perform a patient response activity. Successful completion of the patient response activity can cause the device to suspend or delay a device function, such as administering a treatment to a patient and/or issuing an alert or alarm.

In some examples, the medical device includes monitoring circuitry configured to sense physiological information of a patient. The controller can be configured to detect the patient physiological event based, at least in part, on the sensed physiological information. A patient event can be a temporary physiological problem or abnormality, which can be representative of an underlying patient condition. A patient event can also include injuries and other non-recurring problems that are not representative of underlying physiological condition of the patient. A non-exhaustive list of patient events that can be detected by an external medical device includes, for example: bradycardia, ventricular tachycardia (VT) or ventricular fibrillation (VF), atrial arrhythmias such as premature atrial contractions (PACs), multifocal atrial tachycardia, atrial flutter, and atrial fibrillation, supraventricular tachycardia (SVT), junctional arrhythmias, tachycardia, junctional rhythm, junctional tachycardia, premature junctional contraction, and ventricular arrhythmias such as premature ventricular contractions (PVCs) and accelerated idioventricular rhythm.

In some examples, the device controller is configured to notify the patient of the detection of the one or more events and to receive a patient response to the notification. The patient response can include performing a response activity identifiable by an input component associated with the medical device. In general, the response activity is selected to demonstrate or to provide information about the status of the patient and, in particular, to confirm that the patient is conscious and substantially aware of his or her surroundings. The response activity or activities can also be configured to confirm patient identity (e.g., that the person providing the response is the patient, rather than a bystander or impostor). The response activity can also demonstrate or test a patient ability such as one or more of psychomotor ability, cognitive awareness, and athletic/movement ability. In some examples, the response activity can be a relatively simple action, such as making a simple or reflexive movement in response to a stimulus applied by the device. In other examples, more complex activities, such as providing answers to questions requiring reasoning and logical analysis can be required. The device can be configured to select a particular response activity based on characteristics of the patient and/or the detected patient event.

In some examples, the device can instruct the patient to perform several actions that are each representative of patient ability. In other modes, the device can instruct the patient to perform different types of activities that are representative of different patient abilities. For example, the device can instruct the patient to perform a single activity requiring several patient abilities to complete correctly. Alternatively, the device can instruct the patient to perform a first activity representative of a first patient ability and, once the first activity is correctly completed, to perform a second activity representative of a second patient ability.

This disclosure relates to components, modules, subsystems, circuitry, and/or techniques for use in external medical devices. For example, such components, modules, subsystems, circuitry, and/or techniques can be used in the context of medical devices for providing treatment to and/or monitoring a patient. For example, such medical devices can include monitoring devices configured to monitor a patient to identify occurrence of certain patient events. In some implementations, such devices are capable, in addition to monitoring for patient conditions, of providing treatment to a patient based on detecting a predetermined patient condition.

In some examples, the medical device can be a patient monitoring device, which can be configured to monitor one or more of a patient's physiological parameters without an accompanying treatment component. For example, a patient monitor can include a cardiac monitor for monitoring a patient's cardiac information. Such cardiac information can include, without limitation, heart rate, ECG data, heart sounds data from an acoustic sensor, and other cardiac data. In addition to cardiac monitoring, the patient monitor may perform monitoring of other relevant patient parameters, including glucose levels, blood oxygen levels, lung fluids, lung sounds, and blood pressure.

The devices described herein are capable of continuous, substantially continuous, long-term and/or extended use or wear by, or attachment or connection to, a patient. In this regard, the device may be configured to be used or worn by, or attached or connected to, a patient, without substantial interruption, for example, up to hours or beyond (e.g., weeks, months, or even years). For example, in some implementations, such a period of use or wear may be at least 4 hours. For example, such a period of use or wear may be at least 24 hours or one day. For example, such a period of use or wear may be at least 7 days. For example, such a period of use or wear may be at least one month. In some implementations, such devices can be removed for a period of time before use, wear, attachment, or connection to the patient is resumed, e.g., to change batteries, to change or wash the garment, and/or to take a shower. Similarly, the device can be configured for continuous, substantially continuous, long-term and/or extended monitoring of one or more patient physiological conditions. For instance, in addition to cardiac monitoring, the medical device may be capable of monitoring a patient for other physiological conditions. Accordingly, in implementations, the device may be configured to monitor blood oxygen, temperature, glucose levels, sleep apnea, snoring and/or other sleep conditions, heart sounds, lung sounds, tissue fluids, etc. using a variety of sensors including radio frequency (RF) sensors, ultrasonic sensors, electrodes, etc. In some instances, the device can carry out its monitoring in periodic or aperiodic time intervals or times. For example, the monitoring during intervals or times can be triggered by a patient action or another event. For example, one or more durations between periodic or aperiodic intervals or times can be patient and/or other non-patient user configurable.

In some examples, the device 10 can output a defibrillation therapy in the form of a biphasic pulse of between about 0 and 150 Amps. For example, the biphasic waveform is a biphasic truncated exponential waveform. The device 10 can be programmed to provide between about 75 joules to about 150 joules (±5%) at 20° C. (68° F.) when discharged into a 50 ohm resistive load. In implementations, settings within that range can be programmable in 25 joule increments. In an implementation, the device can be configured to deliver about 35 Amps for a maximum joule defibrillating shock delivered into a 50 ohm load. In some examples, the defibrillation shock sequence can comprise about 1 pulse to about 10 pulses. In some examples, the sequence can comprise about 5 pulses. If conversion of the arrhythmia occurs after a shock, the device automatically precludes delivery of remaining shocks in the sequence. With respect to pacing therapy, in implementations, a maximum current level of current waveform can range from 0 mAmps to about 200 mAmps. In some examples, a pulse width can range from about 0.05 ms to about 2 ms. In some examples, a frequency of the pulses can range from about 30 pulses per minute (PPM) to about 200 PPM. In accordance with one implementation, a 40 ms square wave pulse can be used.

Before the delivery of the therapeutic electrical pulses, electrically conductive fluid is ejected from the openings 117 (see FIG. 22) in the therapy electrodes through the mesh interface and onto the patient's skin. In some examples, as shown in FIG. 19, the therapy electrode can include a plurality of electrically conductive fluid reservoirs 121 (e.g., in some examples, about 2 to about 10 gel reservoirs, or 2 to 10 reservoirs, or about 10 to about 20 reservoirs, or 10 to 20 reservoirs, or about 20 to about 100 reservoirs, or 20 to 100 reservoirs) disposed thereon. Each of the plurality of reservoirs 121 includes a predetermined quantity of electrically conductive fluid, e.g., in some examples, about 0.1 cubic-centimeter (cc) to about 2 cc, or about 0.1 cc to about 2 cc, or about 1 cc to about 20 cc, or about 2 cc to about 20 cc, or 2 cc to 20 cc, or about 20 cc to about 50 cc, or 20 cc to 50 cc.

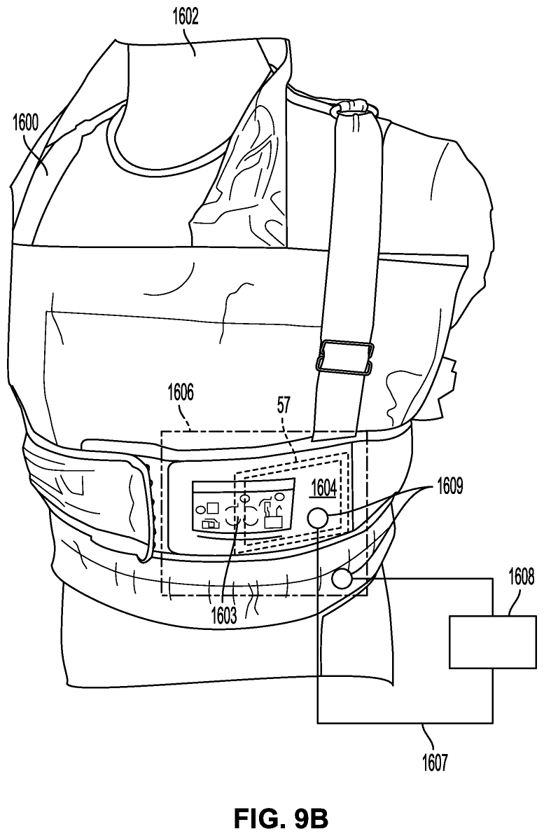

With reference to FIGS. 11A and 11B, an example of the medical device controller 14 is illustrated. The controller 14 can be powered by a rechargeable battery 212. The rechargeable battery 212 can be removable from a housing 206 of the medical device controller 14 to enable a patient and/or caregiver to swap a depleted (or near-depleted) battery 212 for a charged battery. The controller 14 includes a patient interface such as a touch screen 220 that can provide information to the patient, caregiver, and/or bystanders.

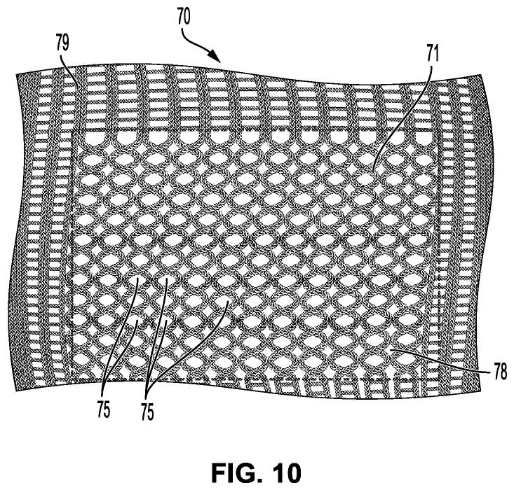

With reference to FIG. 12, a schematic example of the medical device controller 14 is illustrated. As shown in FIG. 12, the controller 14 includes at least one processor 318, a patient interface manager 314, a sensor interface 312, an optional therapy delivery interface 302, data storage 304 (which can include patient data storage 316), an optional network interface 306, a patient interface 308 (e.g., including the touch screen 220 shown in FIGS. 11A and 11B), and a battery 310. The sensor interface 312 can be coupled to any one or combination of sensors to receive information indicative of cardiac activity. For example, the sensor interface 312 can be coupled to one or more sensing devices including, for example, sensing electrodes 328, contact sensors 330, pressure sensors 332, accelerometers or motion sensors 334, and radio frequency (RF)-energy based sensors 331 (e.g., tissue fluid sensors). The controller 14 can also include an optical sensor 336, such as a digital camera, for capturing static or video images of the device surroundings. Although designs from different vendors are different, a digital camera usually consists of a charge-coupled device (CCD) or complementary metal-oxide-semiconductor (CMOS) imaging sensor, a lens, a multifunctional video control chip, and a set of discrete components (e.g., capacitor, resistors, and connectors). The therapy delivery interface 302 (if included) can be coupled to one or more electrodes that provide therapy to the patient including, for example, one or more therapy electrodes 320, pacing electrodes 322, and/or TENS electrodes 324. The sensor interface 312 and the therapy delivery interface 302 can implement a variety of coupling and communication techniques for facilitating the exchange of data between the sensors and/or therapy delivery devices and the controller 14.

The medical device controller 14 can comprise one or more input components configured to receive a response input from the patient. The input components can comprise at least one of: the response button 210; the touch screen 220; an audio detection device, such as a microphone 338; the motion sensor 334; the contact sensor 330; the pressure sensor 332; a gesture recognitions component, such as the optical sensor 336; or a patient physiological sensor, such as the sensing electrodes 328.

In some examples, the medical device controller 14 includes a cardiac event detector 326 to monitor the cardiac activity of the patient and identify cardiac events experienced by the patient based on received cardiac signals. In other examples, cardiac event detection can be performed using algorithms for analyzing patient ECG signals obtained from the sensing electrodes 328. Additionally, the cardiac event detector 326 can access patient templates (e.g., which can be stored in the data storage 304 as patient data 316) that can assist the cardiac event detector 326 in identifying cardiac events experienced by the particular patient (e.g., by performing template matching algorithms).

The at least one processor 318 can perform a series of instructions that control the operation of the other components of the controller 14. In some examples, the patient interface manager 314 is implemented as a software component that is stored in the data storage 304 and executed by the at least one processor 318 to control, for example, the patient interface component 308. The patient interface manager 314 can control various output components and/or devices of the medical device controller 300 (e.g., patient interface 220 and/or patient interface pod 140 shown in FIG. 12) to communicate with external entities consistent with various acts and/or display screens described herein. For example, such output components and/or devices can include speakers, tactile and/or vibration output elements, visual indicators, monitors, displays, LCD screens, LEDs, Braille output elements, and the like. Additionally, the patient interface manager 314 can be integrated with the treatment-providing components of the controller 14 so that the patient can control and, in some cases, suspend, delay, or cancel treatment using the patient interface.

Although a wearable medical device and a support garment for such a device have been described in detail for the purpose of illustration based on what is currently considered to be the most practical examples, it is to be understood that such detail is solely for that purpose and that the subject matter of this disclosure is not limited to the disclosed examples, but, on the contrary, is intended to cover modifications and equivalent arrangements. For example, it is to be understood that this disclosure contemplates that, to the extent possible, one or more features of any example can be combined with one or more features of any other example.

1. A mesh interface for use with a support garment of a wearable cardiac therapeutic device, the mesh interface comprising:
a first side comprising hydrophobic dielectric fiber(s) proximate to one or more electrically conductive fluid deployment openings on a therapy electrode;
a second side comprising hydrophilic dielectric fiber(s) proximate to the patient's skin; and
conductive fiber(s) and/or conductive particles configured to be interspersed with the hydrophobic dielectric fiber(s) and with the hydrophilic dielectric fiber(s), such that the conductive fiber(s) and/or conductive particles conduct therapeutic electrical current from the therapy electrode to the patient's skin,
wherein the mesh interface is configured to facilitate transfer of electrically conductive fluid from one or more electrically conductive fluid reservoirs disposed on the therapy electrode through the one or more electrically conductive fluid deployment openings of the therapy electrode and towards the patient's skin.

2. The mesh interface according to claim 1, wherein the hydrophobic dielectric fiber(s) are configured to facilitate movement of and/or pull the electrically conductive fluid from the one or more electrically conductive fluid deployment openings of the therapy electrode.

3. The mesh interface according to claim 1, wherein the hydrophilic dielectric fiber(s) are configured to facilitate movement of and/or push the electrically conductive fluid towards the patient's skin.

4. The mesh interface according to claim 1, wherein the hydrophobic dielectric fiber(s) are selected from the group consisting of polyester, polypropylene, olefin, acrylic, modacrylic, silk, hydrophobic nylon, wool, spandex, bamboo, and combinations thereof.

5. (canceled)

6. The mesh interface according to claim 1, wherein the hydrophobic dielectric fiber(s) comprise at least one hydrophobic coating and/or at least one hydrophobic impregnant.

7. (canceled)

8. (canceled)

9. The mesh interface according to claim 1, wherein the hydrophilic dielectric fiber(s) are selected from the group consisting of cotton, wool, linen, acetate, cellulosic, rayon, hydrophilic nylon, polyester and combinations thereof.

10. (canceled)

11. The mesh interface according to claim 1, wherein the hydrophilic dielectric fiber(s) comprise at least one hydrophilic coating and/or at least one hydrophilic impregnate.

12.-15. (canceled)

16. The mesh interface according to claim 1, wherein the conductive fiber(s) and/or conductive particles are configured to form a plurality of conductive pathways extending from the first side of the mesh interface to the second side of the mesh interface.

17. The mesh interface according to claim 1, wherein the mesh interface further comprises opening(s) extending through the mesh interface from the first side to the second side, the mesh interface being configured to facilitate transfer of the electrically conductive fluid from the at least one therapy electrode to the patient's skin via the opening(s).

18. The mesh interface according to claim 17, wherein at least a portion of the opening(s) are aligned with respective electrically conductive fluid deployment opening(s) on the therapy electrode to facilitate transfer of the electrically conductive fluid from the at least one therapy electrode to the patient's skin via the opening(s).

19.-27. (canceled)

28. A support garment of a wearable cardiac therapeutic device, the support garment comprising the mesh interface according to claim 1.

29.-48. (canceled)

49. A wearable cardiac therapeutic device for improved skin comfort when worn by a patient, the device comprising:
at least one therapy electrode configured to deliver therapeutic electrical pulses to a patient's heart; and
a support garment configured to support the at least one therapy electrode in electrical communication with the patient's body, the support garment comprising:
at least one support pocket disposed on an inside surface of the support garment for supporting the at least one therapy electrode on the support garment; and
a mesh interface formed as part of the at least one support pocket, the mesh interface configured to facilitate electrical contact between the at least one therapy electrode and the patient's skin,
wherein the mesh interface comprises:
a first side comprising hydrophobic dielectric fiber(s) proximate to one or more electrically conductive fluid deployment openings on a therapy electrode;
a second side comprising hydrophilic dielectric fiber(s) proximate to the patient's skin; and
conductive fiber(s) and/or conductive particles configured to be interspersed with the hydrophobic dielectric fiber(s) and with the hydrophilic dielectric fiber(s) such that the conductive fiber(s) and/or conductive particles conduct therapeutic electrical current from the therapy electrode to the patient's skin,
wherein the mesh interface is configured to facilitate transfer of electrically conductive fluid from one or more electrically conductive fluid reservoirs disposed on the therapy electrode through the one or more electrically conductive fluid deployment openings of the therapy electrode and towards the patient's skin.

50. The wearable cardiac therapeutic device according to claim 49, wherein the hydrophobic dielectric fiber(s) are configured to facilitate movement of and/or pull the electrically conductive fluid from the one or more electrically conductive fluid deployment openings of the therapy electrode.

51. The wearable cardiac therapeutic device according to claim 49, wherein the hydrophilic dielectric fiber(s) are configured to facilitate movement of and/or push the electrically conductive fluid towards the patient's skin.

52. The wearable cardiac therapeutic device according to claim 49, wherein the hydrophobic dielectric fiber(s) are selected from the group consisting of polyester, polypropylene, olefin, acrylic, modacrylic, silk, hydrophobic nylon, wool, spandex, bamboo, and combinations thereof.

53. (canceled)

54. The wearable cardiac therapeutic device according to claim 49, wherein the hydrophobic dielectric fiber(s) comprise at least one hydrophobic coating and/or at least one hydrophobic impregnant.

55. The wearable cardiac therapeutic device according to claim 49, wherein the hydrophilic dielectric fiber(s) are selected from the group consisting of cotton, wool, linen, acetate, cellulosic, rayon, hydrophilic nylon, polyester and combinations thereof.

56.-59. (canceled)

60. The wearable cardiac therapeutic device according to claim 49, wherein the conductive fiber(s) and/or conductive particles are configured to form a plurality of conductive pathways extending from the first side of the mesh interface to the second side of the mesh interface.

61. The wearable cardiac therapeutic device according to claim 49, wherein the mesh interface further comprises opening(s) extending through the mesh interface from the first side to the second side, the mesh interface being configured to facilitate transfer of the electrically conductive fluid from the at least one therapy electrode to the patient's skin via the opening(s).

62. The wearable cardiac therapeutic device according to claim 61, wherein at least a portion of the opening(s) are aligned with respective electrically conductive fluid deployment opening(s) on the therapy electrode to facilitate transfer of the electrically conductive fluid from the at least one therapy electrode to the patient's skin via the opening(s).

63.-68. (canceled)

69. A mesh interface for use with a support garment of a wearable cardiac therapeutic device, the mesh interface comprising:
a first side comprising hydrophobic fiber(s) proximate to one or more conductive fluid deployment openings on a therapy electrode;
a second side comprising hydrophilic fiber(s) proximate to the patient's skin; and
conductive fiber(s) and/or conductive particles configured to be interspersed with the hydrophobic fiber(s) and the hydrophilic fiber(s) such that the conductive fiber(s) and/or conductive particles conduct therapeutic electrical current from the therapy electrode to the patient's skin,
wherein the mesh interface is configured to facilitate transfer of electrically conductive fluid from one or more electrically conductive fluid reservoirs disposed on the therapy electrode through the one or more electrically conductive fluid deployment openings of the therapy electrode and towards the patient's skin.

70. A mesh interface for use with a support garment of a wearable cardiac therapeutic device, the mesh interface comprising:
a first side comprising fiber(s) proximate to one or more conductive fluid deployment openings on a therapy electrode;
a second side comprising fiber(s) proximate to the patient's skin, wherein the fiber(s) of the second side are more hydrophilic than the fibers of the first side; and
conductive fiber(s) and/or conductive particles configured to be interspersed with the fiber(s) of the first side and the fiber(s) of the second side such that the conductive fiber(s) and/or conductive particles conduct therapeutic electrical current from the therapy electrode to the patient's skin,
wherein the mesh interface is configured to facilitate transfer of electrically conductive fluid from one or more electrically conductive fluid reservoirs disposed on the therapy electrode through the one or more electrically conductive fluid deployment openings of the therapy electrode and towards the patient's skin.

71. (canceled)
